SEQUENCE LISTING (1 sequence)

SEQ ID NO: 1 - protein, 13 aa, Artificial sequence
  Synthetic construct
KKKGQEEEYVFIE

(19) United States
(12) Patent Application Publication
(10) Pub. No.: US 2025/0262216 A1
(43) Pub. Date: Aug. 21, 2025

(54) THERAPEUTIC USES OF MACROCYCLIC COMPOUNDS

(71) Applicant: TURNING POINT THERAPEUTICS, INC., San Diego, CA (US)

(72) Inventors: Brion William MURRAY, San Diego, CA (US); Dayong ZHAI, San Diego, CA (US); Jingrong J. CUI, San Diego, CA (US)

(21) Appl. No.: 17/905,503

(22) PCT Filed: Mar. 1, 2021
(86) PCT No.: PCT/US2021/020255

§ 371 Date: Sep. 1, 2022

Related U.S. Application Data
(60) Provisional application No. 62/984,159, filed on Mar. 2, 2020.

(51) Int. Cl. A61K 31/529

(52) U.S. Cl. CPC A61K 31/529

(57) ABSTRACT

This disclosure relates to the use of certain diaryl macrocycle compounds in the treatment of disease in mammals. This disclosure also relates to compositions including such compounds, and to methods of using such compositions in the treatment of diseases in mammals, especially in humans.

### CROSS REFERENCE TO RELATED APPLICATIONS

This application is a U.S. National Stage Entry of International Application No. PCT/US2021/020255 filed Mar. 1, 2021, which claims the priority benefit of U.S. Provisional Patent Application Ser. No. 62/984,159, filed Mar. 2, 2020, the entire contents of each are incorporated herein by reference in their entireties.

### REFERENCE TO A “SEQUENCE LISTING”

This application contains a Sequence Listing which has been submitted electronically in ASCII format and is hereby incorporated by reference in its entirety. Said ASCII copy, created on Mar. 26, 2024, is named 2024-03-26_Sequence_Listing_ST25_058438-519N01US.txt and is 525 bytes in size.

### TECHNICAL FIELD

This disclosure relates to the use of certain diaryl macrocycle compounds in the treatment of disease in mammals. This disclosure also relates to compositions including such compounds, and to methods of using such compositions in the treatment of diseases in mammals, especially in humans.

### BACKGROUND

Protein kinases are key regulators for cell growth, proliferation and survival. Genetic and epigenetic alterations accumulate in cancer cells leading to abnormal activation of signal transduction pathways which drive malignant processes. (Manning, G. et al, The protein kinase complement of the human genome. Science 2002, 298, 1912-1934). Pharmacological inhibition of these signaling pathways presents promising intervention opportunities for targeted cancer therapies. (Sawyers, C. Targeted cancer therapy. Nature 2004, 432, 294-297).

MET, also called hepatocyte growth factor receptor (HGFR), was discovered in 1984 (Cooper, C. S., et al Molecular cloning of a new transforming gene from a chemically transformed human cell line. Nature 1984, 311, 29-33). Hepatocyte growth factor (HGF), also known as scatter factor (SF), is the high-affinity natural ligand of MET (Bottaro D P et al. Identification of the hepatocyte growth factor receptor as the c-met proto-oncogene product. Science 1991, 251 (4995), 802-804). The HGF/MET signaling pathway is implicated in invasive growth during embryo development, postnatal organ regeneration, wound healing and tissue regeneration processes. However, the HGF/MET axis is frequently hijacked by cancer cells for tumorigenesis, invasive growth, and metastasis (Boccaccio, C.; Comoglio, P. M. Invasive growth: a MET-driven generic programme for cancer and stem cells. Nat. Rev. Cancer 2006, 6, 637-645). Deregulations of MET and/or HGF via activating mutations, gene amplifications, overexpression, and both autocrine or paracrine loop regulation influence cell growth, proliferation, angiogenesis, invasion, survival, and metastasis, leading to tumorigenesis and tumor progression (Ma, P C et al. Expression and mutational analysis of MET in human solid cancers. Genes Chromosomes Cancer 2008, 47, 1025-1037). Over-expression of MET and/or HGF has been detected in a large variety of solid tumors such as liver, breast, pancreas, lung, kidney, bladder, ovary, brain, prostate, and many others, and is often associated with a metastatic phenotype and poor prognosis (Maulik, G., et al. Role of the hepatocyte growth factor receptor, MET, in oncogenesis and potential for therapeutic inhibition. Cytokine Growth Factor Rev. 2002, 13, 41-59). MET amplification has been reported in different human cancers including gastroesophageal carcinomas, colorectal cancers, NSCLC, medulloblastomas, and glioblastomas (Smolen, G. A., et al. Amplification of MET may identify a subset of cancers with extreme sensitivity to the selective tyrosine kinase inhibitor PHA-665752. Proc. Natl. Acad. Sci. U.S.A 2006, 103, 2316-2321). A diverse set of MET mutations in the tyrosine kinase domain, juxtamembrane, and extracellular domain of both germline and somatic mutations have been described in many solid tumors, including hereditary and sporadic human papillary renal carcinomas, lung cancer, ovarian cancer, childhood hepatocellular carcinomas, squamous cell carcinoma of the head and neck, and gastric cancer (Ghiso, E.; Giordano, S. Targeting MET: why, where and how?Curr. Opin. Pharmacol. 2013, 13, 511-518). MET exon 14 deletion represents a novel class of actionable oncogenic event with potential clinical impact and therapeutic applications in patients affected by different cancer types (Pilotto S, MET exon 14 juxtamembrane splicing mutations: clinical and therapeutical perspectives for cancer therapy. Ann Transl Med. 2017 5(1):2). Autocrine or paracrine stimulation is one mechanism for aberrant MET activation. The MET autocrine activation plays a causal role in the development of malignant melanoma and acquisition of the metastatic phenotype (Otsuka, T., et al. MET autocrine activation induces development of malignant melanoma and acquisition of the metastatic phenotype. Cancer Res. 1998, 58, 5157-5167). For glioblastoma (GBM), HGF autocrine expression correlated with MET phosphorylation levels in HGF autocrine cell lines, and showed high sensitivity to MET inhibition in vivo, while an HGF paracrine environment could enhance glioblastoma growth in vivo but did not demonstrated sensitivity to MET inhibition (Xie, Q., et al. Hepatocyte growth factor (HGF) autocrine activation predicts sensitivity to MET inhibition in glioblastoma. Proc. Natl. Acad. Sci. U.S.A 2012, 109, 570-575). The aberrant expression of HGF is a crucial element in AML pathogenesis that leads to autocrine activation of MET in nearly half of the AML cell lines and clinical samples (Kentsis, A., et al. Autocrine activation of the MET receptor tyrosine kinase in acute myeloid leukemia. Nat. Med. 2012, 18, 1118-1122).

Upregulation of HGF/MET signaling has been frequently reported as compensatory signaling to confer resistance for kinase targeted therapies. MET amplification has been detected in 4%-20% of NSCLC patients with the EGFR mutations who acquired resistance to gefitinib or erlotinib treatment (Sequist, L. V., et al. Analysis of tumor specimens at the time of acquired resistance to EGFR-TKI therapy in 155 patients with EGFR-mutant lung cancers. Clin. Cancer Res. 2013, 19, 2240-2247). Upregulation of ligand HGF represents another mechanism of EGFR-TKI resistance. High HGF expression was discovered among clinical specimens with acquired resistance that did not have a T790M mutation or MET amplification as well as among cases that exhibited primary resistance despite having EGFR-TKI sensitive activating EGFR gene mutations (Yano, S., et al. Hepatocyte growth factor induces gefitinib resistance of lung adenocarcinoma with epidermal growth factor receptor-activating mutations. Cancer Res. 2008, 68, 9479-9487). Amplification of MET is associated with acquired resistance to cetuximab or panitumumab in metastatic colorectal cancer patients that do not develop KRAS mutations during anti-EGFR therapy (Bardelli, A., et al. Amplification of the MET Receptor Drives Resistance to Anti-EGFR Therapies in Colorectal Cancer. Cancer Discov. 2013, 3, 658-673). Growth factor-driven resistance from tumor microenvironment represents a potential common mechanism for anticancer kinase inhibitors. The upregulation of stromal HGF confers resistance to the BRAF inhibitor ramurafenib in BRAF-mutant melanoma cells (Straussman, R., et al. Tumour micro-environment elicits innate resistance to RAF inhibitors through HGF secretion. Nature 2012, 487, 500-504). It was reported that ligand-mediated activation of alternative receptor tyrosine kinases was observed in cancer cells originally dependent on either MET, FGFR2, or FGFR3, and RTKs from the HER and EGFR families as well as MET compensated for loss of each other (Harbinski, F., et al. Rescue screens with secreted proteins reveal compensatory potential of receptor tyrosine kinases in driving cancer growth. Cancer Discov. 2012, 2, 948-959). Therefore, blocking adaptive cellular responses that drive compensatory ligand expression is necessary for achieving optimal and sustained antitumor effects.

Genomic alterations to the receptor tyrosine kinase are oncogenic drivers for a range of cancers (Campbell et al 2016 Nat Genet 48, 607-16; Sadiq et al., 2013 J Clin Oncol 31, 1089-96) as well as resistance mechanisms to other molecular medicines such as osimertinib for the treatment of lung cancer patients (Ko et al., 2017 Ann Transl Med 5(1), 4; Liu et al., 2018 Molecular Cancer 17, article 53). Patients treated with MET targeted therapies often develop drug resistance by either bypass signaling, mutation to MET, or by unknown mechanisms as is illustrated by a recent publication (Recondo et al, 2020 Clin Cancer Res, doi 10.1158/1078-0432.CCR-19-3608). In the Recondo study, 35% of the 20 patients treated with a MET targeted therapy had MET mutations at disease progression (e.g. MET residues H1094, G1163, L1195, D1228, Y1230, and high levels of MET amplification). As such, inhibitors that potently inhibit MET or potently inhibit mutated forms of MET are expected to have enhanced clinical benefit for patients with amplified MET or mutated forms of MET.

Src is a non-receptor tyrosine kinase that is deregulated in many types of cancer, and a key downstream transducer of many RTKs, including EGFR, HER2, and c-Met. Activation of Src signaling has been implicated in conferring therapeutic resistance to targeted antiendocrine therapies, receptor tyrosine kinase therapies, traditional chemotherapies, and radiation therapies. (Zhang S, et al Trends Pharmacol Sci. 2012, 33, 122). Src inhibitor may play important roles in combinatorial regimens in overcoming resistance to current anticancer therapies and in preventing metastatic recurrence. Cytoplasmic tyrosine kinases (also known as non-receptor tyrosine kinases) of the Src family (SFKs) play important roles in signal transduction induced by a large number of extracellular stimuli including growth factors and integrins. Elevated SFK activity is found in more than 80% of human colorectal cancer (CRC) and this has been associated with poor clinical outcome. (Summy J M, et al. Cancer Metastasis Rev. 2003, 22, 337-358) The SFK member Yes regulates specific oncogenic signalling pathways important for colon cancer progression that is not shared with c-Src. (Scancier F. et al. PLoS One. 2011, 6(2): e17237) WASF2-FGR fusion genes were found in lung squamous carcinoma, ovarian serous cystadenocarcinoma, and skin cutaneous melanoma. (Stransky N, et al. Nature Communications 2014, 5, 4846) Estrogen receptor-positive (ER⁺) breast cancers adapt to hormone deprivation and become resistant to antiestrogen therapy. Mutations in the inhibitory SH2 domain of the SRC family kinase (SFK) LYN were related to ER⁺ tumors that remained highly proliferative after treatment with the aromatase inhibitor letrozole. LYN was upregulated in multiple ER⁺ breast cancer lines resistant to long-term estrogen deprivation. (Schwarz L J, et al. J Clin Invest. 2014, 124, 5490-5502) Therefore, targeting LYN will be a rational strategy overcoming the escape from antiestrogens in a subset of ER⁺ breast cancers. It was reported that LYN was overexpressed in castrate-resistant prostate cancer (CRPC), enhanced AR transcriptional activity, and accelerated CRPC progression, and targeting Lyn kinase induced AR dissociation from the molecular chaperone Hsp90, leading to its ubiquitination and proteasomal degradation. (Zardan A., et al. Oncogenesis 2014, 3, e115) The Lyn tyrosine kinase is a potential therapeutic target for the treatment of CRPC. The Src family kinase FYN is involved in signal transduction pathways in the nervous system, as well as the development and activation of T lymphocytes under normal physiological conditions. Activation of Fyn is observed in various cancers, including melanoma, glioblastoma, squamous cell carcinoma, prostate and breast cancers. (Elias D., et al. Pharmacological Research 2015, 100, 250-254) Fyn was upregulated in tamoxifen-resistant breast cancer cell lines and plays a key role in the resistance mechanism. Peripheral T-cell lymphomas (PTCLs) are a heterogeneous group of aggressive non Hodgkin lymphomas with poor prognosis. FYN activating mutations were found in PTCL, and promoted the growth of cells transformed via expression of activated FYN mutant alleles. SRC kinase inhibitors may play important roles in the treatment of PTCLs. (Couronne L, et al. Blood 2013, 122, 811).

It is desirable to prepare compounds that have activity against disease-driving kinase inhibitors, especially compounds that have activity against genetically altered MET, SCR and CSF1R. New compounds with polypharmacology profiles are also desired for targeting the primary oncogene drivers and their acquired resistance mechanisms including secondary mutations, bypass signaling, MET, cancer stemness, and metastasis.

### SUMMARY

Compounds that inhibit MET, SRC, and CSF1R gene products have been discovered. Compounds of the formula I

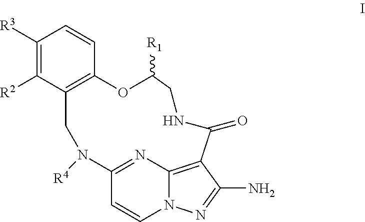

wherein R¹, R², R³, and R⁴ are defined as described herein have been shown to have activity against wild-type and mutant MET, SRC, and CSF1R.

One such compound is (7S)-3-amino-14-ethyl-11-fluoro-7-methyl-4-oxo-4,5,6,7,13,14-hexahydro-1,15-ethenopyrazolo[4,3-f][1,4,8,10]benzoxatriazacyclotridecine-12-carbonitrile (also herein referred to as “Compound 1”), represented by the formula

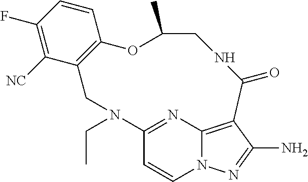 [SMILES: CCN1Cc2c(ccc(F)c2C#N)O[C@@H](C)CNC(=O)c2c(N)nn3ccc1nc23]

has been shown to be a potent small-molecule kinase inhibitor showing activity against wild-type and mutant wild-type and mutant MET, SRC, and CSF1R. Compound 1 has properties, including anti-tumor properties, which are pharmacologically mediated through inhibition of receptor and non-receptor tyrosine kinases. Compounds of the formula I, in particular, Compound 1, are disclosed in International Patent Publication No. WO2019/023417, which is incorporated herein by reference in its entirety.

In one aspect, the present disclosure provide a method of treating disease, such as cancer, in a mammal, in particular a human patient comprising, administering to the mammal, in particular a human patient, a therapeutically effective amount of a compound that inhibits MET, SRC, and CSF1R, wherein the disease is mediated by a genetically altered MET. In some embodiments, the compound that inhibits MET, SRC, and CSF1R is of the formula I

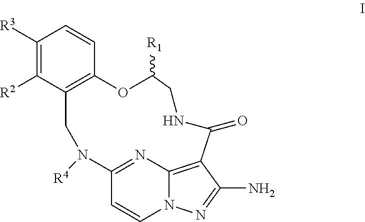

or a pharmaceutically acceptable salt thereof, wherein R¹, R², R³, and R⁴ are defined as described herein. In some embodiments, the compound that inhibits MET, SRC, and CSF1R is of the formula

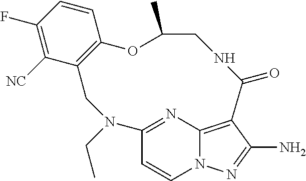 [SMILES: CCN1Cc2c(ccc(F)c2C#N)O[C@@H](C)CNC(=O)c2c(N)nn3ccc1nc23]

or a pharmaceutically acceptable salt thereof. In some embodiments, the mammal, in particular a human patient, has received prior treatment with one or more therapeutic agents.

In another aspect, the present disclosure provides a method of treating cancer in a patient previously shown to express a genetically altered MET comprising, administering to the patient a therapeutically effective amount of a compound that inhibits MET, SRC, and CSF1R. In some embodiments, the compound that inhibits MET, SRC, and CSF1R is of the formula I

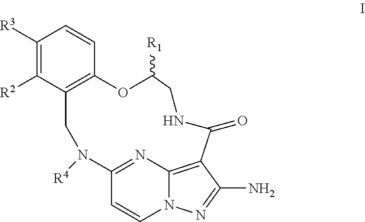

or a pharmaceutically acceptable salt thereof, wherein R¹, R², R³, and R⁴ are defined as described herein. In some embodiments, the compound that inhibits MET, SRC, and CSF1R is of the formula

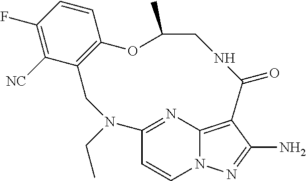 [SMILES: CCN1Cc2c(ccc(F)c2C#N)O[C@@H](C)CNC(=O)c2c(N)nn3ccc1nc23]

or a pharmaceutically acceptable salt thereof. In some embodiments, the patient, has received prior treatment with one or more therapeutic agents.

In another aspect, the present disclosure provides a method of treating cancer in a patient comprising;
i. identifying a genetically altered MET in the patient, and
ii. administering to the patient a therapeutically effective amount of a compound that inhibits MET, SRC, and CSF1R. In some embodiments, the compound that inhibits MET, SRC, and CSF1R is of the formula I

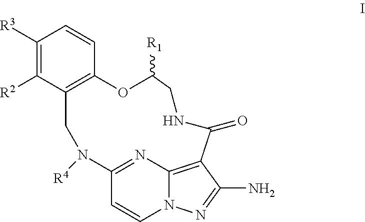

or a pharmaceutically acceptable salt thereof, wherein R¹, R², R³, and R⁴ are defined as described herein. In some embodiments, the compound that inhibits MET, SRC, and CSF1R is of the formula

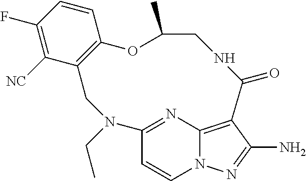 [SMILES: CCN1Cc2c(ccc(F)c2C#N)O[C@@H](C)CNC(=O)c2c(N)nn3ccc1nc23]

or a pharmaceutically acceptable salt thereof. In some embodiments, the patient, has received prior treatment with one or more therapeutic agents.

In another aspect, the present disclosure provides a method of identifying a patient for treatment with a compound that inhibits MET, SRC, and CSF1R. comprising diagnosing the patient with a cancer mediated by a genetically altered MET. In some embodiments, the compound that inhibits MET, SRC, and CSF1R is of the formula I

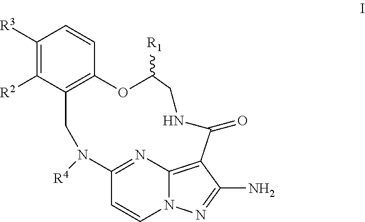

or a pharmaceutically acceptable salt thereof, wherein R¹, R², R³, and R⁴ are defined as described herein. In some embodiments, the compound that inhibits MET, SRC, and CSF1R is of the formula

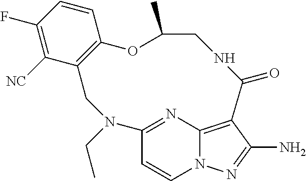 [SMILES: CCN1Cc2c(ccc(F)c2C#N)O[C@@H](C)CNC(=O)c2c(N)nn3ccc1nc23]

or a pharmaceutically acceptable salt thereof. In some embodiments, the patient, has received prior treatment with one or more therapeutic agents.

In another aspect, the present disclosure provides a use of compound that inhibits MET, SRC, and CSF1R in the preparation of a medicament for the treatment of a disease in a patient. In some embodiments, the compound that inhibits MET, SRC, and CSF1R is of the formula I

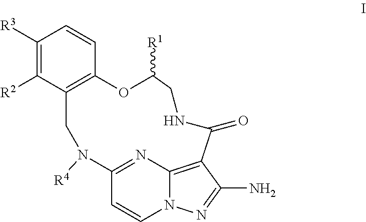

or a pharmaceutically acceptable salt thereof, wherein R¹, R², R³, and R⁴ are defined as described herein. In some embodiments, the compound that inhibits MET, SRC, and CSF1R is of the formula

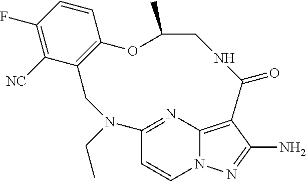 [SMILES: CCN1Cc2c(ccc(F)c2C#N)O[C@@H](C)CNC(=O)c2c(N)nn3ccc1nc23]

or a pharmaceutically acceptable salt thereof. In some embodiments, the patient, has received prior treatment with one or more therapeutic agents.

In another aspect, the present disclosure provides a compound inhibits MET, SRC, and CSF1R for treating cancer in a patient. In some embodiments, the compound that inhibits MET, SRC, and CSF1R is of the formula I

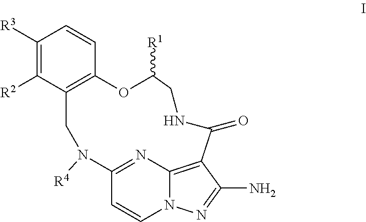

or a pharmaceutically acceptable salt thereof, wherein R¹, R², R³, and R⁴ are defined as described herein. In some embodiments, the compound that inhibits MET, SRC, and CSF1R is of the formula

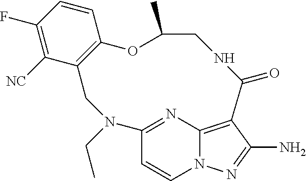 [SMILES: CCN1Cc2c(ccc(F)c2C#N)O[C@@H](C)CNC(=O)c2c(N)nn3ccc1nc23]

or a pharmaceutically acceptable salt thereof. In some embodiments, the patient, has received prior treatment with one or more therapeutic agents.

In another aspect, the present disclosure provides use of a compound that inhibits MET, SRC, and CSF1R for treating cancer in a patient previously shown to express a genetically altered tyrosine or serine/threonine kinase. In some embodiments, the compound that inhibits MET, SRC, and CSF1R is of the formula I

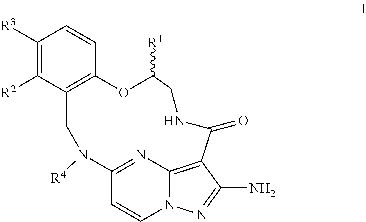

or a pharmaceutically acceptable salt thereof, wherein R¹, R², R³, and R⁴ are defined as described herein. In some embodiments, the compound that inhibits MET, SRC, and CSF1R is of the formula

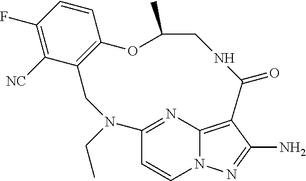 [SMILES: CCN1Cc2c(ccc(F)c2C#N)O[C@@H](C)CNC(=O)c2c(N)nn3ccc1nc23]

or a pharmaceutically acceptable salt thereof. In some embodiments, the patient, has received prior treatment with one or more therapeutic agents.

In another aspect, the present disclosure provide a use a compound inhibits MET, SRC, and CSF1R for treating cancer in a patient, wherein the patient has been previously treated with a cancer therapeutic, and the cancer has developed resistance to the cancer therapeutic. In some embodiments, the compound that inhibits MET, SRC, and CSF1R is of the formula I

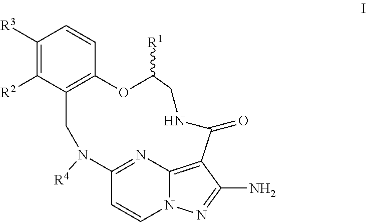

or a pharmaceutically acceptable salt thereof, wherein R¹, R², R³, and R⁴ are defined as described herein. In some embodiments, the compound that inhibits MET, SRC, and CSF1R is of the formula

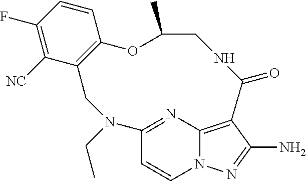 [SMILES: CCN1Cc2c(ccc(F)c2C#N)O[C@@H](C)CNC(=O)c2c(N)nn3ccc1nc23]

or a pharmaceutically acceptable salt thereof. In some embodiments, the patient, has received prior treatment with one or more therapeutic agents.

In another aspect, the present disclosure provides the use of a compound that inhibits MET, SRC, and CSF1R for treating cancer in a patient previously shown to express a genetically altered tyrosine or serine/threonine kinase, wherein the patient has been previously treated with a cancer therapeutic, and the cancer has developed resistance to the cancer therapeutic. In some embodiments, the compound that inhibits MET, SRC, and CSF1R is of the formula I

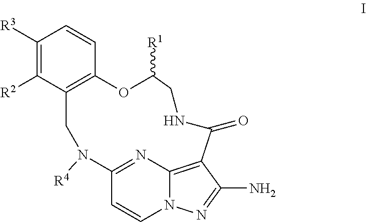

or a pharmaceutically acceptable salt thereof, wherein R¹, R², R³, and R⁴ are defined as described herein. In some embodiments, the compound that inhibits MET, SRC, and CSF1R is of the formula

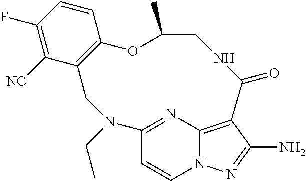 [SMILES: CCN1Cc2c(ccc(F)c2C#N)O[C@@H](C)CNC(=O)c2c(N)nn3ccc1nc23]

or a pharmaceutically acceptable salt thereof. In some embodiments, the patient, has received prior treatment with one or more therapeutic agents.

In some embodiments, the genetically altered MET gene comprising a point mutation that is expressed in the c-Met protein. In some embodiments, the genetically altered MET comprises a point mutation expressed in the c-Met protein at one or more of positions P991, T992, D1010, V1092, H1094, G1163, T1173, H1094, N1100, Y1003, H1106, V1070, V1188, V1092, H1094, G1162, L1195, F1200, V1220, D1228, Y1230, D1231, Y1235, D1246, Y1248, M1250, and M1268. In some embodiments, the genetically altered MET comprises a point mutation expressed in the c-Met protein that is selected from the group consisting of P991S, T992I, D1010H, D1010Y, V1092I, H1094N, H1094R, H1094Y, N1100K, N1100S, Y1003C, Y1003F, Y1003H, H1106D, V1070A, V1092I, V1188I, T1173I, H1094Y, G1163R, L1195F, F1195I, L1195V, F1200I, V1220I, D1228N, D1228H, D1228V, Y1230A, Y1230C, Y1230D, Y1230H, Y1230H, Y1230S, D1231Y, Y1235D, D1246N, D1246H, Y1248D, Y1248H, Y1248C, M1250T, and M1268T. In some embodiments, the genetically altered MET comprises a point mutation expressed in the c-Met protein that is selected from the group consisting of T1173I, P991S, M1250T, T992I, V1092I, F1200I, Y1235D, Y1230H, D1246N, D1246H, Y1248D, Y1248H, Y1248C, and M1268T. In some embodiments, the cancer is exhibiting bypass resistance. In some embodiments, the bypass resistance is mediated by a SRC/CSF1R.

In some embodiments, the cancer is a carcinoma, a sarcoma, a lymphoma, Hodgekin's disease, a melanoma, a mesothelioma, Burkitt's lymphoma, a nasopharyngeal carcinoma, a leukemia, a lung cancer, a breast cancer, a hereditary human papillary renal carcinoma, a sporadic human papillary renal carcinoma, a childhood hepatocellular carcinoma, or a myeloma. In some embodiments, the cancer is selected from the group consisting of ALCL, NSCLC, neuroblastoma, inflammatory myofibroblastic tumor, renal cancer, adult renal cell carcinoma, pediatric renal cell carcinoma, breast cancer, triple negative breast cancer, triple positive breast cancer, HER⁺ breast cancer, mouth cancer, esophageal cancer, laryngeal cancer, pancreatic cancer, bladder cancer, colon cancer, colonic adenocarcinoma, glioblastoma, glioblastoma multiforme, thyroid cancer, anaplastic thyroid cancer, endocrine cancer, bone cancer, cholangiocarcinoma, ovarian cancer, cervical cancer, uterine cancer, testicular cancer, gastric cancer, gastric adenocarcinoma, colorectal cancer, rectal cancer, liver cancer, kidney cancer, angiosarcoma, epithelioid hemangioendothelioma, intrahepatic cholangiocarcinoma, thyroid papillary cancer, spitzoid neoplasms, sarcoma, astrocytoma, brain lower grade glioma, secretory breast carcinoma, mammary analogue carcinoma, acute myeloid leukemia, congenital mesoblastic nephroma, congenital fibrosarcomas, Ph-like acute lymphoblastic leukemia, thyroid carcinoma, skin cancer, head and neck squamous cell carcinoma, pediatric glioma CML, prostate cancer, lung squamous carcinoma, ovarian serous cystadenocarcinoma, skin cutaneous melanoma, metastatic castration-resistant prostate cancer, Hodgkin lymphoma, neuroendocrine tumors, serous and clear cell endometrial cancer.

In some embodiments, the patient has been previously treated with a cancer therapeutic. In some embodiments, the patient has been previously treated with a cancer therapeutic, and the cancer has developed resistance to the cancer therapeutic. In some embodiments, the resistance is a primary intrinsic resistance. In some embodiments, the resistance is an acquired resistance from mutation(s). In some embodiments, the resistance is a bypass resistance. In some embodiments, the resistance is an EMT-based resistance.

Additional embodiments, features, and advantages of the disclosure will be apparent from the following detailed description and through practice of the disclosure. The compounds of the present disclosure can be described as embodiments in any of the following enumerated clauses. It will be understood that any of the embodiments described herein can be used in connection with any other embodiments described herein to the extent that the embodiments do not contradict one another.

1. A method of treating cancer in a patient comprising, administering to the patient a therapeutically effective amount of a compound that inhibits MET, SRC and CSF1R, wherein the cancer is mediated by a genetically altered MET.

2. The method of clause 1, wherein the compound of the formula

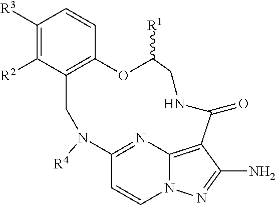

wherein
R¹ is H, deuterium, or C₁-C₆ alkyl;
R² is chloro or —CN;
R³ is H, deuterium, or fluoro;
R⁴ is H or C₁-C₆ alkyl, wherein each hydrogen atom in C₁-C₆ alkyl is independently optionally substituted by deuterium, fluoro, chloro, bromo, —OH, —CN, —OC₁-C₆ alkyl, —NH₂, —NH(C₁-C₆ alkyl), —N(C₁-C₆ alkyl)₂, or C₃-C₇ cycloalkyl, or a pharmaceutically acceptable salt thereof, wherein the cancer is mediated by a genetically altered MET.

3. The method of clause 1 or 2, wherein the genetically altered MET encodes a point mutation expressed in the c-Met protein.

4. The method of any one of clauses 1 to 3, wherein the genetically altered MET encodes a point mutation expressed in the c-Met protein at one or more of positions P991, T992, V1092, H1094, G1163, T1173, L1195, F1200, D1228, Y1230, Y1235, D1246, Y1248, M1250, and M1268.

5. The method of any one of the preceding clauses, wherein the genetically altered MET encodes a point mutation expressed in the c-Met protein that is selected from the group consisting of T1173I, P991S, M1250T, T992I, V1092I, F1200I, Y1235D, Y1230H, D1246N, D1246H, Y1248D, Y1248H, Y1248C, and M1268T.

6. The method of any one of the preceding clauses, wherein the cancer is exhibiting bypass resistance mediated by a SRC/CSF1R.

7. The method of any one of the preceding clauses, wherein R¹ is methyl.

8. The method of any one of the preceding clauses, wherein R² is —CN.

9. The method of any one of the preceding clauses, wherein R³ is fluoro.

10. The method of any one of the preceding clauses, wherein R⁴ is C₁-C₆ alkyl.

11. The method of any one of the preceding clauses, wherein the compound is of the formula

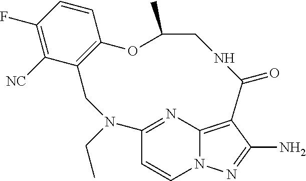 [SMILES: CCN1Cc2c(ccc(F)c2C#N)O[C@@H](C)CNC(=O)c2c(N)nn3ccc1nc23]

or a pharmaceutically acceptable salt thereof.

12. The method of any one of the preceding clauses, wherein the cancer is a carcinoma, a sarcoma, a lymphoma, Hodgekin's disease, a melanoma, a mesothelioma, Burkitt's lymphoma, a nasopharyngeal carcinoma, a leukemia, a lung cancer, a breast cancer, a hereditary human papillary renal carcinoma, a sporadic human papillary renal carcinoma, a childhood hepatocellular carcinoma, or a myeloma.

13. The method of any one of clauses 1 to 12, wherein the cancer is selected from the group consisting of ALCL, NSCLC, neuroblastoma, inflammatory myofibroblastic tumor, renal cancer, adult renal cell carcinoma, pediatric renal cell carcinoma, breast cancer, triple negative breast cancer, triple positive breast cancer, HER breast cancer, mouth cancer, esophageal cancer, laryngeal cancer, pancreatic cancer, bladder cancer, colon cancer, colonic adenocarcinoma, glioblastoma, glioblastoma multiforme, thyroid cancer, anaplastic thyroid cancer, endocrine cancer, bone cancer, cholangiocarcinoma, ovarian cancer, cervical cancer, uterine cancer, testicular cancer, gastric cancer, gastric adenocarcinoma, colorectal cancer, rectal cancer, liver cancer, kidney cancer, angiosarcoma, epithelioid hemangioendothelioma, intrahepatic cholangiocarcinoma, thyroid papillary cancer, spitzoid neoplasms, sarcoma, astrocytoma, brain lower grade glioma, secretory breast carcinoma, mammary analogue carcinoma, acute myeloid leukemia, congenital mesoblastic nephroma, congenital fibrosarcomas, Ph-like acute lymphoblastic leukemia, thyroid carcinoma, skin cancer, head and neck squamous cell carcinoma, pediatric glioma CML, prostate cancer, lung squamous carcinoma, ovarian serous cystadenocarcinoma, skin cutaneous melanoma, metastatic castration-resistant prostate cancer, Hodgkin lymphoma, neuroendocrine tumors, and serous and clear cell endometrial cancer.

14. The method of any one of the preceding clauses, wherein the patient has received prior treatment with one or more therapeutic agents.

15. A method of treating cancer in a patient previously shown to have a cancer mediated by a genetically altered MET comprising, administering to the patient a therapeutically effective amount of a compound that inhibits MET, SRC and CSF1R.

16. The method of clause 15, wherein the compound of the formula

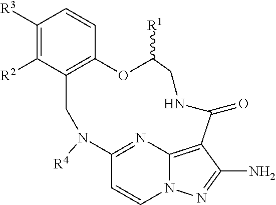

wherein
R¹ is H, deuterium, or C₁-C₆ alkyl;
R² is chloro or —CN;
R³ is H, deuterium, or fluoro;
R⁴ is H or C₁-C₆ alkyl, wherein each hydrogen atom in C₁-C₆ alkyl is independently optionally substituted by deuterium, fluoro, chloro, bromo, —OH, —CN, —OC₁-C₆ alkyl, —NH₂, —NH(C₁-C₆ alkyl), —N(C₁-C₆ alkyl)₂, or C₃-C₇ cycloalkyl, or a pharmaceutically acceptable salt thereof.

17. The method of clause 15 or 16, wherein the genetically altered MET encodes a point mutation expressed in the c-Met protein.

18. The method of any one of clauses 15 to 17, wherein the genetically altered MET encodes a point mutation expressed in the c-Met protein at one or more of positions P991, T992, V1092, H1094, G1163, T1173, L1195, F1200, D1228, Y1230, Y1235, D1246, Y1248, M1250, and M1268.

19. The method of any one of clauses 15 to 18, wherein the genetically altered MET encodes a point mutation expressed in the c-Met protein that is selected from the group consisting of T1173I, P991S, M1250T, T992I, V1092I, F1200I, Y1235D, Y1230H, D1246N, D1246H, Y1248D, Y1248H, Y1248C, and M1268T.

20. The method of any one of clauses 15 to 19, wherein the cancer is exhibiting bypass resistance mediated by a SRC/CSF1R.

21. The method of any one of clauses 15 to 20, wherein R¹ is methyl.

22. The method of any one of clauses 15 to 21, wherein R² is —CN.

23. The method of any one of clauses 15 to 22, wherein R³ is fluoro.

24. The method of any one of clauses 15 to 23, wherein R⁴ is C₁-C₆ alkyl.

25. The method of any one of clauses 15 to 24, wherein the compound is of the formula

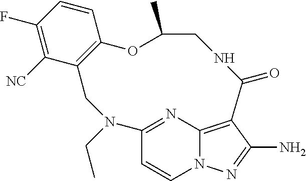 [SMILES: CCN1Cc2c(ccc(F)c2C#N)O[C@@H](C)CNC(=O)c2c(N)nn3ccc1nc23]

or a pharmaceutically acceptable salt thereof.

26. The method of any one of clauses 15 to 25, wherein the cancer is a carcinoma, a sarcoma, a lymphoma, Hodgekin's disease, a melanoma, a mesothelioma, Burkitt's lymphoma, a nasopharyngeal carcinoma, a leukemia, a lung cancer, a breast cancer, a hereditary human papillary renal carcinoma, a sporadic human papillary renal carcinoma, a childhood hepatocellular carcinoma, or a myeloma.

27. The method of any one of clauses 15 to 25, wherein the cancer is selected from the group consisting of ALCL, NSCLC, neuroblastoma, inflammatory myofibroblastic tumor, renal cancer, adult renal cell carcinoma, pediatric renal cell carcinoma, breast cancer, triple negative breast cancer, triple positive breast cancer, HER breast cancer, mouth cancer, esophageal cancer, laryngeal cancer, pancreatic cancer, bladder cancer, colon cancer, colonic adenocarcinoma, glioblastoma, glioblastoma multiforme, thyroid cancer, anaplastic thyroid cancer, endocrine cancer, bone cancer, cholangiocarcinoma, ovarian cancer, cervical cancer, uterine cancer, testicular cancer, gastric cancer, gastric adenocarcinoma, colorectal cancer, rectal cancer, liver cancer, kidney cancer, angiosarcoma, epithelioid hemangioendothelioma, intrahepatic cholangiocarcinoma, thyroid papillary cancer, spitzoid neoplasms, sarcoma, astrocytoma, brain lower grade glioma, secretory breast carcinoma, mammary analogue carcinoma, acute myeloid leukemia, congenital mesoblastic nephroma, congenital fibrosarcomas, Ph-like acute lymphoblastic leukemia, thyroid carcinoma, skin cancer, head and neck squamous cell carcinoma, pediatric glioma CML, prostate cancer, lung squamous carcinoma, ovarian serous cystadenocarcinoma, skin cutaneous melanoma, metastatic castration-resistant prostate cancer, Hodgkin lymphoma, neuroendocrine tumors, and serous and clear cell endometrial cancer.

28. The method of any one of clauses 15 to 27, wherein the patient has received prior treatment with one or more therapeutic agents.

29. A compound that inhibits MET, SRC and CSF1R for treating cancer in a patient comprising, wherein the cancer is mediated by a genetically altered MET.

30. The compound of clause 29 having of the formula

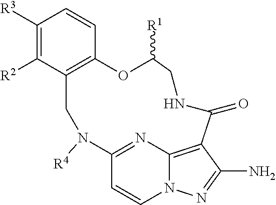

wherein
R¹ is H, deuterium, or C₁-C₆ alkyl;
R² is chloro or —CN;
R³ is H, deuterium, or fluoro;
R⁴ is H or C₁-C₆ alkyl, wherein each hydrogen atom in C₁-C₆ alkyl is independently optionally substituted by deuterium, fluoro, chloro, bromo, —OH, —CN, —OC₁-C₆ alkyl, —NH₂, —NH(C₁-C₆ alkyl), —N(C₁-C₆ alkyl)₂, or C₃-C₇ cycloalkyl, or a pharmaceutically acceptable salt thereof, for treating cancer in a patient comprising, wherein the cancer is mediated by a genetically altered MET.

31. The compound of clause 29 or 30, wherein the genetically altered MET encodes a point mutation expressed in the c-Met protein.

32. The compound of any one of clauses 29 to 31, wherein the genetically altered MET encodes comprises a point mutation expressed in the c-Met protein at one or more of positions P991, T992, V1092, H1094, G1163, T1173, L1195, F1200, D1228, Y1230, Y1235, D1246, Y1248, M1250, and M1268.

33. The compound of any one of clauses 29 to 32, wherein the genetically altered MET encodes a point mutation expressed in the c-Met protein that is selected from the group consisting of T1173I, P991S, M1250T, T992I, V1092I, F1200I, Y1235D, Y1230H, D1246N, D1246H, Y1248D, Y1248H, Y1248C, and M1268T.

34. The compound of any one of clauses 29 to 33, wherein the cancer is exhibiting bypass resistance mediated by a SRC/CSF1R.

35. The compound of any one of clauses 29 to 34, wherein R¹ is methyl.

36. The compound of any one of clauses 29 to 35, wherein R² is —CN.

37. The compound of any one of clauses 29 to 36, wherein R³ is fluoro.

38. The compound of any one of clauses 29 to 37, wherein R⁴ is C₁-C₆ alkyl.

39. The compound of any one of clauses 29 to 38, wherein the compound is of the formula

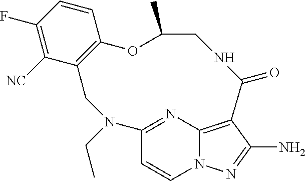 [SMILES: CCN1Cc2c(ccc(F)c2C#N)O[C@@H](C)CNC(=O)c2c(N)nn3ccc1nc23]

or a pharmaceutically acceptable salt thereof.

40. The compound of any one of clauses 29 to 39, wherein the cancer is a carcinoma, a sarcoma, a lymphoma, Hodgekin's disease, a melanoma, a mesothelioma, Burkitt's lymphoma, a nasopharyngeal carcinoma, a leukemia, a lung cancer, a breast cancer, a hereditary human papillary renal carcinoma, a sporadic human papillary renal carcinoma, a childhood hepatocellular carcinoma, or a myeloma.

41. The compound of any one of clauses 29 to 39, wherein the cancer is selected from the group consisting of ALCL, NSCLC, neuroblastoma, inflammatory myofibroblastic tumor, renal cancer, adult renal cell carcinoma, pediatric renal cell carcinoma, breast cancer, triple negative breast cancer, triple positive breast cancer, HER breast cancer, mouth cancer, esophageal cancer, laryngeal cancer, pancreatic cancer, bladder cancer, colon cancer, colonic adenocarcinoma, glioblastoma, glioblastoma multiforme, thyroid cancer, anaplastic thyroid cancer, endocrine cancer, bone cancer, cholangiocarcinoma, ovarian cancer, cervical cancer, uterine cancer, testicular cancer, gastric cancer, gastric adenocarcinoma, colorectal cancer, rectal cancer, liver cancer, kidney cancer, angiosarcoma, epithelioid hemangioendothelioma, intrahepatic cholangiocarcinoma, thyroid papillary cancer, spitzoid neoplasms, sarcoma, astrocytoma, brain lower grade glioma, secretory breast carcinoma, mammary analogue carcinoma, acute myeloid leukemia, congenital mesoblastic nephroma, congenital fibrosarcomas, Ph-like acute lymphoblastic leukemia, thyroid carcinoma, skin cancer, head and neck squamous cell carcinoma, pediatric glioma CML, prostate cancer, lung squamous carcinoma, ovarian serous cystadenocarcinoma, skin cutaneous melanoma, metastatic castration-resistant prostate cancer, Hodgkin lymphoma, neuroendocrine tumors, and serous and clear cell endometrial cancer.

42. The compound of any one of clauses 29 to 41, wherein the patient has received prior treatment with one or more therapeutic agents.

43. Use of a compound that inhibits MET, SRC and CSF1R in the preparation of a medicament for treating cancer in a patient, wherein the cancer is mediated by a genetically altered MET.

44. The use of clause 43, wherein the compound is of the formula

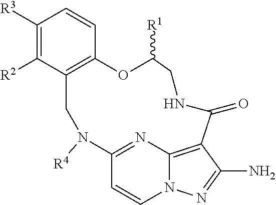

wherein
R¹ is H, deuterium, or C₁-C₆ alkyl;
R² is chloro or —CN;
R³ is H, deuterium, or fluoro;
R⁴ is H or C₁-C₆ alkyl, wherein each hydrogen atom in C₁-C₆ alkyl is independently optionally substituted by deuterium, fluoro, chloro, bromo, —OH, —CN, —OC₁-C₆ alkyl, —NH₂, —NH(C₁-C₆ alkyl), —N(C₁-C₆ alkyl)₂, or C₃-C₇ cycloalkyl, or a pharmaceutically acceptable salt thereof, in the preparation of a medicament for treating cancer in a patient comprising, wherein the cancer is mediated by a genetically altered MET.

45. The use of clause 43 or 44, wherein the genetically altered MET encodes a point mutation expressed in the c-Met protein.

46. The use of any one of clauses 43 to 45, wherein the genetically altered MET encodes a point mutation expressed in the c-Met protein at one or more of positions P991, T992, V1092, H1094, G1163, T1173, L1195, F1200, D1228, Y1230, Y1235, D1246, Y1248, M1250, and M1268.

47. The use of any one of clauses 43 to 46, wherein the genetically altered MET encodes a point mutation expressed in the c-Met protein that is selected from the group consisting of T1173I, P991S, M1250T, T992I, V1092I, F1200I, Y1235D, Y1230H, D1246N, D1246H, Y1248D, Y1248H, Y1248C, and M1268T.

48. The use of any one of clauses 43 to 47, wherein the cancer is exhibiting bypass resistance mediated by a SRC/CSF1R.

49. The use of any one of clauses 43 to 48, wherein R¹ is methyl.

50. The use of any one of clauses 43 to 49, wherein R² is —CN.

51. The use of any one of clauses 43 to 50, wherein R³ is fluoro.

52. The use of any one of clauses 43 to 51, wherein R⁴ is C₁-C₆ alkyl.

53. The use of any one of clauses 43 to 52, wherein the compound is of the formula

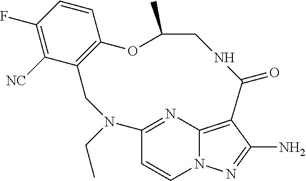 [SMILES: CCN1Cc2c(ccc(F)c2C#N)O[C@@H](C)CNC(=O)c2c(N)nn3ccc1nc23]

or a pharmaceutically acceptable salt thereof.

54. The use of any one of clauses 43 to 53, wherein the cancer is a carcinoma, a sarcoma, a lymphoma, Hodgekin's disease, a melanoma, a mesothelioma, Burkitt's lymphoma, a nasopharyngeal carcinoma, a leukemia, a lung cancer, a breast cancer, a hereditary human papillary renal carcinoma, a sporadic human papillary renal carcinoma, a childhood hepatocellular carcinoma, or a myeloma.

55. The use of any one of clauses 43 to 53, wherein the cancer is selected from the group consisting of ALCL, NSCLC, neuroblastoma, inflammatory myofibroblastic tumor, renal cancer, adult renal cell carcinoma, pediatric renal cell carcinoma, breast cancer, triple negative breast cancer, triple positive breast cancer, HER breast cancer, mouth cancer, esophageal cancer, laryngeal cancer, pancreatic cancer, bladder cancer, colon cancer, colonic adenocarcinoma, glioblastoma, glioblastoma multiforme, thyroid cancer, anaplastic thyroid cancer, endocrine cancer, bone cancer, cholangiocarcinoma, ovarian cancer, cervical cancer, uterine cancer, testicular cancer, gastric cancer, gastric adenocarcinoma, colorectal cancer, rectal cancer, liver cancer, kidney cancer, angiosarcoma, epithelioid hemangioendothelioma, intrahepatic cholangiocarcinoma, thyroid papillary cancer, spitzoid neoplasms, sarcoma, astrocytoma, brain lower grade glioma, secretory breast carcinoma, mammary analogue carcinoma, acute myeloid leukemia, congenital mesoblastic nephroma, congenital fibrosarcomas, Ph-like acute lymphoblastic leukemia, thyroid carcinoma, skin cancer, head and neck squamous cell carcinoma, pediatric glioma CML, prostate cancer, lung squamous carcinoma, ovarian serous cystadenocarcinoma, skin cutaneous melanoma, metastatic castration-resistant prostate cancer, Hodgkin lymphoma, neuroendocrine tumors, and serous and clear cell endometrial cancer.

56. The method of any one of clauses 43 to 55, wherein the patient has received prior treatment with one or more therapeutic agents.

57. A medicament comprising a compound that inhibits MET, SRC and CSF1R for use in a method of treating cancer in a patient comprising, wherein the cancer is mediated by a genetically altered MET.

58. The medicament of clause 57, wherein the compound is of the formula

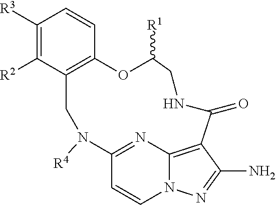

wherein
R¹ is H, deuterium, or C₁-C₆ alkyl;
R² is chloro or —CN;
R³ is H, deuterium, or fluoro;
R⁴ is H or C₁-C₆ alkyl, wherein each hydrogen atom in C₁-C₆ alkyl is independently optionally substituted by deuterium, fluoro, chloro, bromo, —OH, —CN, —OC₁-C₆ alkyl, —NH₂, —NH(C₁-C₆ alkyl), —N(C₁-C₆ alkyl)₂, or C₃-C₇ cycloalkyl, or a pharmaceutically acceptable salt thereof, for use in a method of treating cancer in a patient comprising, wherein the cancer is mediated by a genetically altered MET.

59. The medicament of clause 57 or 58, wherein the genetically altered MET encodes a point mutation expressed in the c-Met protein.

60. The medicament of any one of clauses 57 to 59, wherein the genetically altered MET encodes a point mutation expressed in the c-Met protein at one or more of positions P991, T992, V1092, H1094, G1163, T1173, L1195, F1200, D1228, Y1230, Y1235, D1246, Y1248, M1250, and M1268.

61. The medicament of any one of clauses 57 to 60, wherein the genetically altered MET encodes a point mutation expressed in the c-Met protein that is selected from the group consisting of T1173I, P991S, M1250T, T992I, V1092I, F1200I, Y1235D, Y1230H, D1246N, D1246H, Y1248D, Y1248H, Y1248C, and M1268T.

62. The medicament of any one of clauses 57 to 61, wherein the cancer is exhibiting bypass resistance mediated by a SRC/CSF1R.

63. The medicament of any one of clauses 57 to 62, wherein R¹ is methyl.

64. The medicament of any one of clauses 57 to 63, wherein R² is —CN.

65. The medicament of any one of clauses 57 to 64, wherein R³ is fluoro.

66. The medicament of any one of clauses 57 to 65, wherein R⁴ is C₁-C₆ alkyl.

67. The medicament of any one of clauses 57 to 66, wherein the compound is of the formula

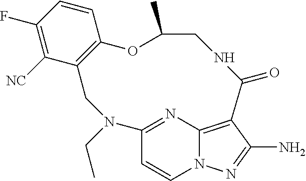 [SMILES: CCN1Cc2c(ccc(F)c2C#N)O[C@@H](C)CNC(=O)c2c(N)nn3ccc1nc23]

or a pharmaceutically acceptable salt thereof.

68. The medicament of any one of clauses 57 to 67, wherein the cancer is a carcinoma, a sarcoma, a lymphoma, Hodgekin's disease, a melanoma, a mesothelioma, Burkitt's lymphoma, a nasopharyngeal carcinoma, a leukemia, a lung cancer, a breast cancer, a hereditary human papillary renal carcinoma, a sporadic human papillary renal carcinoma, a childhood hepatocellular carcinoma, or a myeloma.

69. The medicament of any one of clauses 57 to 68, wherein the cancer is selected from the group consisting of ALCL, NSCLC, neuroblastoma, inflammatory myofibroblastic tumor, renal cancer, adult renal cell carcinoma, pediatric renal cell carcinoma, breast cancer, triple negative breast cancer, triple positive breast cancer, HER breast cancer, mouth cancer, esophageal cancer, laryngeal cancer, pancreatic cancer, bladder cancer, colon cancer, colonic adenocarcinoma, glioblastoma, glioblastoma multiforme, thyroid cancer, anaplastic thyroid cancer, endocrine cancer, bone cancer, cholangiocarcinoma, ovarian cancer, cervical cancer, uterine cancer, testicular cancer, gastric cancer, gastric adenocarcinoma, colorectal cancer, rectal cancer, liver cancer, kidney cancer, angiosarcoma, epithelioid hemangioendothelioma, intrahepatic cholangiocarcinoma, thyroid papillary cancer, spitzoid neoplasms, sarcoma, astrocytoma, brain lower grade glioma, secretory breast carcinoma, mammary analogue carcinoma, acute myeloid leukemia, congenital mesoblastic nephroma, congenital fibrosarcomas, Ph-like acute lymphoblastic leukemia, thyroid carcinoma, skin cancer, head and neck squamous cell carcinoma, pediatric glioma CML, prostate cancer, lung squamous carcinoma, ovarian serous cystadenocarcinoma, skin cutaneous melanoma, metastatic castration-resistant prostate cancer, Hodgkin lymphoma, neuroendocrine tumors, and serous and clear cell endometrial cancer.

70. The medicament of any one of clauses 57 to 69, wherein the patient has received prior treatment with one or more therapeutic agents.

71. Use of a compound that inhibits MET, SRC and CSF1R in a method of treating cancer in a patient comprising, wherein the cancer is mediated by a genetically altered MET.

72. The use of clause 71, wherein the compound is of the formula

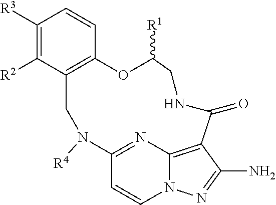

wherein
R¹ is H, deuterium, or C₁-C₆ alkyl;
R² is chloro or —CN;
R³ is H, deuterium, or fluoro;
R⁴ is H or C₁-C₆ alkyl, wherein each hydrogen atom in C₁-C₆ alkyl is independently optionally substituted by deuterium, fluoro, chloro, bromo, —OH, —CN, —OC₁-C₆ alkyl, —NH₂, —NH(C₁-C₆ alkyl), —N(C₁-C₆ alkyl)₂, or C₃-C₇ cycloalkyl, or a pharmaceutically acceptable salt thereof, in a method of treating cancer in a patient comprising, wherein the cancer is mediated by a genetically altered MET.

73. The use of clause 71 or 72, wherein the genetically altered MET encodes a point mutation expressed in the c-Met protein.

74. The use of any one of clauses 71 to 73, wherein the genetically altered MET encodes a point mutation expressed in the c-Met protein at one or more of positions P991, T992, V1092, H1094, G1163, T1173, L1195, F1200, D1228, Y1230, Y1235, D1246, Y1248, M1250, and M1268.

75. The use of any one of clauses 71 to 74, wherein the genetically altered MET encodes a point mutation expressed in the c-Met protein that is selected from the group consisting of T1173I, P991S, M1250T, T992I, V1092I, F1200I, Y1235D, Y1230H, D1246N, D1246H, Y1248D, Y1248H, Y1248C, and M1268T.

76. The use of any one of clauses 71 to 75, wherein the cancer is exhibiting bypass resistance mediated by a SRC/CSF1R.

77. The use of any one of clauses 71 to 76, wherein R¹ is methyl.

78. The use of any one of clauses 71 to 77, wherein R² is —CN.

79. The use of any one of clauses 71 to 78, wherein R³ is fluoro.

80. The use of any one of clauses 71 to 79, wherein R⁴ is C₁-C₆ alkyl.

81. The use of any one of clauses 71 to 80, wherein the compound is of the formula

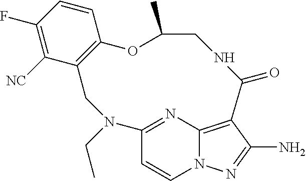 [SMILES: CCN1Cc2c(ccc(F)c2C#N)O[C@@H](C)CNC(=O)c2c(N)nn3ccc1nc23]

or a pharmaceutically acceptable salt thereof.

82. The use of any one of clauses 71 to 81, wherein the cancer is a carcinoma, a sarcoma, a lymphoma, Hodgekin's disease, a melanoma, a mesothelioma, Burkitt's lymphoma, a nasopharyngeal carcinoma, a leukemia, a lung cancer, a breast cancer, a hereditary human papillary renal carcinoma, a sporadic human papillary renal carcinoma, a childhood hepatocellular carcinoma, or a myeloma.

83. The use of any one of clauses 71 to 81, wherein the cancer is selected from the group consisting of ALCL, NSCLC, neuroblastoma, inflammatory myofibroblastic tumor, renal cancer, adult renal cell carcinoma, pediatric renal cell carcinoma, breast cancer, triple negative breast cancer, triple positive breast cancer, HER breast cancer, mouth cancer, esophageal cancer, laryngeal cancer, pancreatic cancer, bladder cancer, colon cancer, colonic adenocarcinoma, glioblastoma, glioblastoma multiforme, thyroid cancer, anaplastic thyroid cancer, endocrine cancer, bone cancer, cholangiocarcinoma, ovarian cancer, cervical cancer, uterine cancer, testicular cancer, gastric cancer, gastric adenocarcinoma, colorectal cancer, rectal cancer, liver cancer, kidney cancer, angiosarcoma, epithelioid hemangioendothelioma, intrahepatic cholangiocarcinoma, thyroid papillary cancer, spitzoid neoplasms, sarcoma, astrocytoma, brain lower grade glioma, secretory breast carcinoma, mammary analogue carcinoma, acute myeloid leukemia, congenital mesoblastic nephroma, congenital fibrosarcomas, Ph-like acute lymphoblastic leukemia, thyroid carcinoma, skin cancer, head and neck squamous cell carcinoma, pediatric glioma CML, prostate cancer, lung squamous carcinoma, ovarian serous cystadenocarcinoma, skin cutaneous melanoma, metastatic castration-resistant prostate cancer, Hodgkin lymphoma, neuroendocrine tumors, and serous and clear cell endometrial cancer.

84. The use of any one of clauses 71 to 83, wherein the patient was previously shown to express a c-Met comprising a mutation encoded by a genetically altered MET.

85. The use of any one of clauses 71 to 84, wherein the patient has received prior treatment with one or more therapeutic agents.

86. A method of treating cancer in a patient comprising;
i. identifying a genetically altered MET in the patient, and
ii. administering to the patient a therapeutically effective amount of a compound that inhibits MET, SRC and CSF1R.

87. The method of clause 86, wherein the compound is of the formula

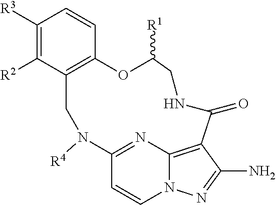

wherein
R¹ is H, deuterium, or C₁-C₆ alkyl;
R² is chloro or —CN;
R³ is H, deuterium, or fluoro;
R⁴ is H or C₁-C₆ alkyl, wherein each hydrogen atom in C₁-C₆ alkyl is independently optionally substituted by deuterium, fluoro, chloro, bromo, —OH, —CN, —OC₁-C₆ alkyl, —NH₂, —NH(C₁-C₆ alkyl), —N(C₁-C₆ alkyl)₂, or C₃-C₇ cycloalkyl, or a pharmaceutically acceptable salt thereof.

88. The method of clause 86 or 87, wherein the genetically altered MET encodes a point mutation expressed in the c-Met protein.

89. The method of any one of clauses 86 to 88, wherein the genetically altered MET encodes a point mutation expressed in the c-Met protein at one or more of positions P991, T992, V1092, H1094, G1163, T1173, L1195, F1200, D1228, Y1230, Y1235, D1246, Y1248, M1250, and M1268.

90. The method of any one of clauses 86 to 89, wherein the genetically altered MET encodes a point mutation expressed in the c-Met protein that is selected from the group consisting of T1173I, P991S, M1250T, T992I, V1092I, F1200I, Y1235D, Y1230H, D1246N, D1246H, Y1248D, Y1248H, Y1248C, and M1268T.

91. The method of any one of clauses 86 to 90, wherein the cancer is exhibiting bypass resistance mediated by a SRC/CSF1R.

92. The method of any one of clauses 86 to 91, wherein R¹ is methyl.

93. The method of any one of clauses 86 to 92, wherein R² is —CN.

94. The method of any one of clauses 86 to 93, wherein R³ is fluoro.

95. The method of any one of clauses 86 to 94, wherein R⁴ is C₁-C₆ alkyl.

96. The method of any one of clauses 86 to 95, wherein the compound is of the formula

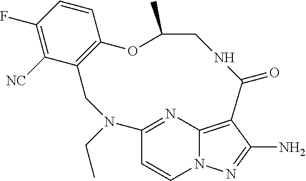 [SMILES: CCN1Cc2c(ccc(F)c2C#N)O[C@@H](C)CNC(=O)c2c(N)nn3ccc1nc23]

or a pharmaceutically acceptable salt thereof.

97. The method of any one of clauses 86 to 96, wherein the cancer is a carcinoma, a sarcoma, a lymphoma, Hodgekin's disease, a melanoma, a mesothelioma, Burkitt's lymphoma, a nasopharyngeal carcinoma, a leukemia, a lung cancer, a breast cancer, a hereditary human papillary renal carcinoma, a sporadic human papillary renal carcinoma, a childhood hepatocellular carcinoma, or a myeloma.

98. The method of any one of clauses 86 to 96, wherein the cancer is selected from the group consisting of ALCL, NSCLC, neuroblastoma, inflammatory myofibroblastic tumor, renal cancer, adult renal cell carcinoma, pediatric renal cell carcinoma, breast cancer, triple negative breast cancer, triple positive breast cancer, HER breast cancer, mouth cancer, esophageal cancer, laryngeal cancer, pancreatic cancer, bladder cancer, colon cancer, colonic adenocarcinoma, glioblastoma, glioblastoma multiforme, thyroid cancer, anaplastic thyroid cancer, endocrine cancer, bone cancer, cholangiocarcinoma, ovarian cancer, cervical cancer, uterine cancer, testicular cancer, gastric cancer, gastric adenocarcinoma, colorectal cancer, rectal cancer, liver cancer, kidney cancer, angiosarcoma, epithelioid hemangioendothelioma, intrahepatic cholangiocarcinoma, thyroid papillary cancer, spitzoid neoplasms, sarcoma, astrocytoma, brain lower grade glioma, secretory breast carcinoma, mammary analogue carcinoma, acute myeloid leukemia, congenital mesoblastic nephroma, congenital fibrosarcomas, Ph-like acute lymphoblastic leukemia, thyroid carcinoma, skin cancer, head and neck squamous cell carcinoma, pediatric glioma CML, prostate cancer, lung squamous carcinoma, ovarian serous cystadenocarcinoma, skin cutaneous melanoma, metastatic castration-resistant prostate cancer, Hodgkin lymphoma, neuroendocrine tumors, and serous and clear cell endometrial cancer.

99. The method of any one of clauses 86 to 98, wherein the patient has received prior treatment with one or more therapeutic agents.

100. The method of any one of clauses 86 to 99, wherein the step of identifying comprises subjecting a patient sample to a test selected from the group consisting of FISH, IHC, PCR and gene sequencing.

101. A method of identifying a patient for treatment with a compound that inhibits MET, SRC and CSF1R comprising diagnosing the patient with a cancer mediated by a genetically altered MET.

102. The method of clause 101, wherein the compound is of the formula

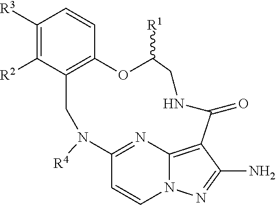

wherein
R¹ is H, deuterium, or C₁-C₆ alkyl;
R² is chloro or —CN;
R³ is H, deuterium, or fluoro;
R⁴ is H or C₁-C₆ alkyl, wherein each hydrogen atom in C₁-C₆ alkyl is independently optionally substituted by deuterium, fluoro, chloro, bromo, —OH, —CN, —OC₁-C₆ alkyl, —NH₂, —NH(C₁-C₆ alkyl), —N(C₁-C₆ alkyl)₂, or C₃-C₇ cycloalkyl, or a pharmaceutically acceptable salt thereof.

103. The method of clause 101 or 102, wherein the genetically altered MET encodes a point mutation expressed in the c-Met protein.

104. The method of any one of clauses 101 to 103, wherein the genetically altered MET encodes a point mutation expressed in the c-Met protein at one or more of positions P991, T992, V1092, H1094, G1163, T1173, L1195, F1200, D1228, Y1230, Y1235, D1246, Y1248, M1250, and M1268.

105. The method of any one of clauses 101 to 104, wherein the genetically altered MET encodes a point mutation expressed in the c-Met protein that is selected from the group consisting of T1173I, P991S, M1250T, T992I, V1092I, F1200I, Y1235D, Y1230H, D1246N, D1246H, Y1248D, Y1248H, Y1248C, and M1268T.

106. The method of any one of clauses 101 to 105, wherein the cancer is exhibiting bypass resistance mediated by a SRC/CSF1R.

107. The method of any one of clauses 101 to 106, wherein R¹ is methyl.

108. The method of any one of clauses 101 to 107, wherein R² is —CN.

109. The method of any one of clauses 101 to 108, wherein R³ is fluoro.

110. The method of any one of clauses 101 to 109, wherein R⁴ is C₁-C₆ alkyl.

111. The method of any one of clauses 101 to 110, wherein the compound is of the formula

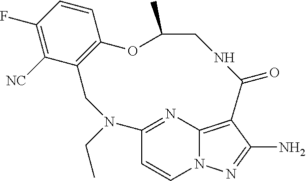 [SMILES: CCN1Cc2c(ccc(F)c2C#N)O[C@@H](C)CNC(=O)c2c(N)nn3ccc1nc23]

or a pharmaceutically acceptable salt thereof.

112. The method of any one of clauses 101 to 111, wherein the cancer is a carcinoma, a sarcoma, a lymphoma, Hodgekin's disease, a melanoma, a mesothelioma, Burkitt's lymphoma, a nasopharyngeal carcinoma, a leukemia, a lung cancer, a breast cancer, a hereditary human papillary renal carcinoma, a sporadic human papillary renal carcinoma, a childhood hepatocellular carcinoma, or a myeloma.

113. The method of any one of clauses 101 to 111, wherein the cancer is selected from the group consisting of ALCL, NSCLC, neuroblastoma, inflammatory myofibroblastic tumor, renal cancer, adult renal cell carcinoma, pediatric renal cell carcinoma, breast cancer, triple negative breast cancer, triple positive breast cancer, HER breast cancer, mouth cancer, esophageal cancer, laryngeal cancer, pancreatic cancer, bladder cancer, colon cancer, colonic adenocarcinoma, glioblastoma, glioblastoma multiforme, thyroid cancer, anaplastic thyroid cancer, endocrine cancer, bone cancer, cholangiocarcinoma, ovarian cancer, cervical cancer, uterine cancer, testicular cancer, gastric cancer, gastric adenocarcinoma, colorectal cancer, rectal cancer, liver cancer, kidney cancer, angiosarcoma, epithelioid hemangioendothelioma, intrahepatic cholangiocarcinoma, thyroid papillary cancer, spitzoid neoplasms, sarcoma, astrocytoma, brain lower grade glioma, secretory breast carcinoma, mammary analogue carcinoma, acute myeloid leukemia, congenital mesoblastic nephroma, congenital fibrosarcomas, Ph-like acute lymphoblastic leukemia, thyroid carcinoma, skin cancer, head and neck squamous cell carcinoma, pediatric glioma CML, prostate cancer, lung squamous carcinoma, ovarian serous cystadenocarcinoma, skin cutaneous melanoma, metastatic castration-resistant prostate cancer, Hodgkin lymphoma, neuroendocrine tumors, and serous and clear cell endometrial cancer.

114. The method of any one of clauses 101 to 113, wherein the patient has received prior treatment with one or more therapeutic agents.

115. The method of any one of clauses 101 to 114, wherein the patient is identified by subjecting a patient sample to a test selected from the group consisting of FISH, IHC, PCR and gene sequencing.

116. The method of any one of clauses 101 to 114, wherein the diagnosing comprises obtaining a sample from a patient, and modifying the sample using a biological test or biological assay selected from the group consisting of FISH, IHC, PCR and gene sequencing, to provide a measured result showing a genetically altered MET in the sample.

### DETAILED DESCRIPTION

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the terms “including,” “containing,” and “comprising” are used in their open, non-limiting sense.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Concentrations that are given as percentages refer to mass ratios, unless indicated differently.

Except as otherwise noted, the methods and techniques of the present embodiments are generally performed according to conventional methods well known in the art and as described in various general and more specific references that are cited and discussed throughout the present disclosure.

### Definitions

As used herein, the term “alkyl” includes a chain of carbon atoms, which is optionally branched and contains from 1 to 20 carbon atoms. It is to be further understood that in certain embodiments, alkyl may be advantageously of limited length, including C₁-C₁₂, C₁-C₁₀, C₁-C₉, C₁-C₈, C₁-C₇, C₁-C₆, and C₁-C₄, Illustratively, such particularly limited length alkyl groups, including C₁-C₅, C₁-C₇, C₁-C₆, and C₁-C₄, and the like may be referred to as “lower alkyl.” Illustrative alkyl groups include, but are not limited to, methyl, ethyl, n-propyl, isopropyl, n-butyl, isobutyl, sec-butyl, tert-butyl, pentyl, 2-pentyl, 3-pentyl, neopentyl, hexyl, heptyl, octyl, and the like. Alkyl may be substituted or unsubstituted. Typical substituent groups include cycloalkyl, aryl, heteroaryl, heteroalicyclic, hydroxy, alkoxy, aryloxy, mercapto, alkylthio, arylthio, cyano, halo, carbonyl, oxo, (═O), thiocarbonyl, O-carbamyl, N-carbamyl, O-thiocarbamyl, N-thiocarbamyl, C-amido, N-amido, C-carboxy, O-carboxy, nitro, and amino, or as described in the various embodiments provided herein. It will be understood that “alkyl” may be combined with other groups, such as those provided above, to form a functionalized alkyl. By way of example, the combination of an “alkyl” group, as described herein, with a “carboxy” group may be referred to as a “carboxyalkyl” group. Other non-limiting examples include hydroxyalkyl, aminoalkyl, and the like.

As used herein, the term “cycloalkyl” refers to a 3 to 15 member all-carbon monocyclic ring, including an all-carbon 5-member/6-member or 6-member/6-member fused bicyclic ring, or a multicyclic fused ring (a “fused” ring system means that each ring in the system shares an adjacent pair of carbon atoms with each other ring in the system) group, where one or more of the rings may contain one or more double bonds but the cycloalkyl does not contain a completely conjugated pi-electron system. It will be understood that in certain embodiments, cycloalkyl may be advantageously of limited size such as C₃-C₁₃, C₃-C₉, C₃-C₆ and C₄-C₆. Cycloalkyl may be unsubstituted, or substituted as described for alkyl or as described in the various embodiments provided herein. Illustrative cycloalkyl groups include, but are not limited to, cyclopropyl, cyclobutyl, cyclopentyl, cyclopentenyl, cyclopentadienyl, cyclohexyl, cyclohexenyl, cycloheptyl, adamantyl, norbornyl, norbornenyl, 9H-fluoren-9-yl, and the like. Illustrative examples of cycloalkyl groups shown in graphical representations include the following entities, in the form of properly bonded moieties:

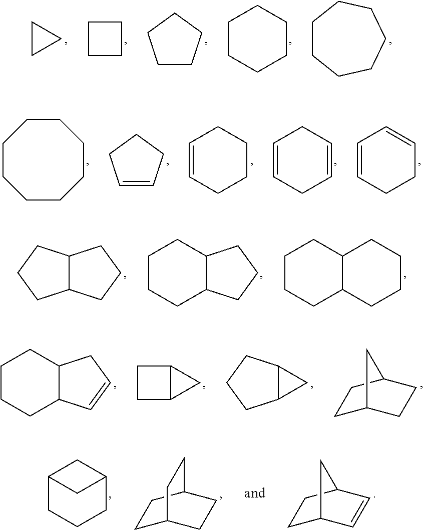

As used herein, “hydroxy” or ““hydroxyl” refers to an —OH group.

As used herein, “halo” or “halogen” refers to fluorine, chlorine, bromine or iodine.

As used herein, “cyano” refers to a —CN group.

The term “substituted” means that the specified group or moiety bears one or more substituents. The term “unsubstituted” means that the specified group bears no substituents. Where the term “substituted” is used to describe a structural system, the substitution is meant to occur at any valency-allowed position on the system. In some embodiments, “substituted” means that the specified group or moiety bears one, two, or three substituents. In other embodiments, “substituted” means that the specified group or moiety bears one or two substituents. In still other embodiments, “substituted” means the specified group or moiety bears one substituent.

As used herein, “optional” or “optionally” means that the subsequently described event or circumstance may but need not occur, and that the description includes instances where the event or circumstance occurs and instances in which it does not. For example, “wherein each hydrogen atom in C₁-C₆ alkyl” means that a substituent may be but need not be present on the C₁-C₆ alkyl by replacement of a hydrogen atom for each substituent group, and the description includes situations where the C₁-C₆ alkyl is substituted and situations where the C₁-C₆ alkyl is not substituted.

As used herein, the term “pharmaceutically acceptable salt” refers to those salts which counter ions which may be used in pharmaceuticals. See, generally, S. M. Berge, et al., “Pharmaceutical Salts,” J. Pharm. Sci., 1977, 66, 1-19. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt. Such salts include:
(1) acid addition salts, which can be obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methane sulfonic acid, ethanesulfonic acid, p-toluenesulfonic acid, salicylic acid, tartaric acid, citric acid, succinic acid or malonic acid and the like; or
(2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, trimethamine, N-methylglucamine, and the like.

Pharmaceutically acceptable salts are well known to those skilled in the art, and any such pharmaceutically acceptable salt may be contemplated in connection with the embodiments described herein. Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington's Pharmaceutical Sciences, 17th Edition, Mack Publishing Company, Easton, Pa., 1985.

For a compound of Formula I that contains a basic nitrogen, a pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, or ethanesulfonic acid, or any compatible mixture of acids such as those given as examples herein, and any other acid and mixture thereof that are regarded as equivalents or acceptable substitutes in light of the ordinary level of skill in this technology.

The disclosure also relates to pharmaceutically acceptable prodrugs of the compounds of Formula I, and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula I). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

The present disclosure also relates to pharmaceutically active metabolites of compounds of Formula I, and uses of such metabolites in the methods of the disclosure. A “pharmaceutically active metabolite” means a pharmacologically active product of metabolism in the body of a compound of Formula I or salt thereof. Prodrugs and active metabolites of a compound may be determined using routine techniques known or available in the art. See, e.g., Bertolini et al., J. Med. Chem. 1997, 40, 2011-2016; Shan et al., J. Pharm. Sci. 1997, 86 (7), 765-767; Bagshawe, Drug Dev. Res. 1995, 34, 220-230; Bodor, Adv. Drug Res. 1984, 13, 255-331; Bundgaard, Design of Prodrugs (Elsevier Press, 1985); and Larsen, Design and Application of Prodrugs, Drug Design and Development (Krogsgaard-Larsen et al., eds., Harwood Academic Publishers, 1991).

Any formula depicted herein is intended to represent a compound of that structural formula as well as certain variations or forms. For example, a formula given herein is intended to include a racemic form, or one or more enantiomeric, diastereomeric, or geometric isomers, or a mixture thereof. Additionally, any formula given herein is intended to refer also to a hydrate, solvate, or polymorph of such a compound, or a mixture thereof. Additionally, any formula given herein is intended to refer also to a hydrate, solvate, or polymorph of such a compound, or a mixture thereof. For example, it will be appreciated that compounds depicted by a structural formula containing the symbol “ [symbol] ” include both stereoisomers for the carbon atom to which the symbol “ [symbol] ” is attached, specifically both the bonds “ [symbol] ” and “ [symbol] ” are encompassed by the meaning of “ [symbol] ”.

As used herein, the term “genetically altered” refers to a permanent alteration in the DNA sequence that makes up a gene that can result in a change in the protein sequence encoded by the gene. A gene that is “genetically altered” as described herein, can possess changes in DNA sequence, and/or protein sequence encoded by the DNA sequence, that range in size; for example, a single nucleotide (a.k.a. a single nucleotide polymorphism, SNP or point mutation), a multiple nucleotide polymorphism (MNPs), a large segment of a chromosome that includes multiple genes, such as a gene fusion, and the like. Examples of gene fusions include, but are not limited to, those which are the result of a chromosomal inversion in which a portion of a chromosomal DNA encoding one or more genes rearranges to provide a fusion of two genes not ordinarily in communication in the DNA sequence, chromosomal deletion in which part of a DNA sequence of a chromosome is deleted to provide a fusion of two genes not ordinarily in communication in the DNA sequence, or those which are the result of a translocation in which a portion of chromosomal DNA is spliced and inserted into the same or a different chromosome to provide a fusion of two genes not ordinarily in communication in the DNA sequence. One of skill in the art will readily appreciate that such gene fusions can be found in multiple variants depending on the individual in which the gene fusion has occurred, and each of such variants is contemplated by the methods described herein.

A “genetically altered” gene, or the protein encoded by such gene, can occur as hereditary mutations which can be inherited from a parent and are sometimes referred to as germline mutations, or a “genetically altered” gene, or the protein encoded by such gene, can occur as an acquired (or somatic) mutation that occurs at some point during a person's life. In some instances, a “genetically altered” gene can be described as a de novo (new) mutation, and can be either hereditary or somatic. It will be further understood that “genetically altered” can refer to a situation in which more than one of the changes in DNA sequence described herein can occur in a patient simultaneously, such as a SNP (or point mutation) and a translocation. Such situations can arise from, but are not solely the result of, so-called “acquired resistance” in which a patient having been treated with a kinase inhibitor can develop a mutation in the DNA sequence that reduces the effectiveness of the treatment. Non-limiting examples of such acquired resistance mutations include the point mutations P991S, T992I, V1092I, T1173I, F1200I, D1228N, D1228H, Y1230A, Y1230C, Y1230D, Y1230H, Y1235D, D1246N, D1246H, Y1248D, Y1248H, Y1248C, M1250T, and M1268T in the c-Met protein that is encoded by a genetically altered MET.

As used herein, the term “intrinsic resistance” refers to the pre-existing resistance of disease cells, especially cancer cells, to drug treatment, especially chemotherapy treatment. It will be appreciated that intrinsic resistance can result in resistance of the cells to a single drug, a small group of structurally related drugs, or a several drugs of differing chemical structure (so-called “multidrug resistance” or “MDR”). (Monti, E. 2007. Molecular Determinants of Intrinsic Multidrug Resistance in Cancer Cells and Tumors In B. Teicher (Ed.), Cancer Drug Resistance (pp. 241-260). Totowa, New Jersey: Humana Press Inc.). It will be appreciated that intrinsic resistance can be the result of one or more host-related factors and/or the genetic make-up of the cells. Such factors include but are not limited to immunomodulation; pharmacogenetic factors such as failure to achieve optimal serum drugs levels due to altered ADME or low tolerance to drug-induced side effects; restricted drug access to the tumor site; and microenvironmental cues. Such genetic make-up factors include, but are not limited to altered expression of drug transporters; qualitative alterations of drug target(s); quantitative alterations of drug target(s); changes in intracellular drug handling/metabolism; changes in DNA repair activities, and alteration in apoptotic pathways. (Gottesman, M. M., Annu. Rev. Med., 2002, 53, 516-527).

As used herein, the term “disease” includes, but is not limited to, cancer, pain, inflammatory diseases, such as allergy, asthma, autoimmune diseases, coeliac disease, glomerulonephritis, hepatitis, inflammatory bowel disease (e.g. ulcerative colitis), pre-perfusion injury, transplant rejection, psoriasis, and rheumatoid arthritis; polycythemia vera, essential thrombocythemia, and myeloid metaplasia with myelofibrosis.

As used herein, the term “cancer” includes, but is not limited to, cancers such as carcinomas, sarcomas, lymphomas, Hodgekin's disease, melanomas, mesotheliomas, Burkitt's lymphoma, nasopharyngeal carcinomas, leukemias, lung cancers, breast cancers, hereditary human papillary renal carcinomas, sporadic human papillary renal carcinomas, childhood hepatocellular carcinomas, myeloma, and the like. Examples of “cancers” in connection with the present disclosure include, but are not limited to, ALCL, lung cancer, such as non-small cell lung cancer (NSCLC), including adenocarcinoma, lung squamous cell carcinoma, large cell carcinoma, and large cell neuroendocrine tumors, small cell lung cancer (SCLC), neuroblastoma, inflammatory myofibroblastic tumor, renal cancer, adult renal cell carcinoma, pediatric renal cell carcinoma, breast cancer, such as luminal A, luminal B, triple negative breast cancer, triple positive breast cancer, HER2+, and the like, mouth cancer, colonic adenocarcinoma, glioblastoma, glioblastoma multiforme, thyroid cancer, such as anaplastic thyroid cancer, cholangiocarcinoma, ovarian cancer, gastric cancer, such as gastric adenocarcinoma, colorectal cancer (CRC), angiosarcoma, epithelioid hemangioendothelioma, intrahepatic cholangiocarcinoma, thyroid papillary cancer, spitzoid neoplasms, sarcoma, astrocytoma, brain lower grade glioma, secretory breast carcinoma, mammary analogue carcinoma, acute myeloid leukemia, congenital mesoblastic nephroma, congenital fibrosarcomas, Ph-like acute lymphoblastic leukemia, thyroid carcinoma, skin cancer, such as skin cutaneous melanoma, head and neck squamous cell carcinoma (HNSCC), pediatric glioma CML, prostate cancer, ovarian serous cystadenocarcinoma, skin cutaneous melanoma, castrate-resistant prostate cancer, Hodgkin lymphoma, uterine cancer, such as serous and clear cell endometrial cancer, endometrial cancer, and the like, oral cancer, endocrine cancer, esophageal cancer, laryngeal cancer, pancreatic cancer, colon cancer, bladder cancer, bone cancer, cervical cancer, testicular cancer, rectal cancer, kidney cancer, liver cancer, neuroendocrine tumors, and stomach cancer. It will be appreciated that the term “cancer” includes both primary cancers or primary tumors and metastatic cancers or metastatic tumors, and includes all stages of cancer as known in the art. For example, metastatic NSCLC, metastatic CRC, metastatic pancreatic cancer, metastatic colorectal carcinoma, metastatic HNSCC, metastatic uterine cancer, and the like. It will be appreciated that the term “cancer” includes cancers that involve the upregulation of certain genes or genetic mutations in certain genes that can lead to disease progression, such as small GTPases (e.g. KRAS and the like) and receptor tyrosine kinases such as MET, and the like.

### Representative Embodiments

In some embodiments, the methods described herein relate to the treatment of disease comprising administering to a patient in need of treatment a therapeutically effective amount of a compound having activity against MET, SRC, and CSF1R. In some embodiments, the compound has activity against a genetically altered MET, SRC, and SCF1R. In some embodiments, the compound is of the formula I

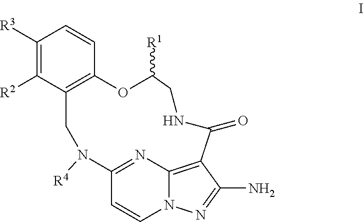

wherein
R¹ is H, deuterium, or C₁-C₆ alkyl;
R² is chloro or —CN;
R³ is H, deuterium, or fluoro;
R⁴ is H or C₁-C₆ alkyl, wherein each hydrogen atom in C₁-C₆ alkyl is independently optionally substituted by deuterium, fluoro, chloro, bromo, —OH, —CN, —OC₁-C₆ alkyl, —NH₂, —NH(C₁-C₆ alkyl), —N(C₁-C₆ alkyl)₂, or C₃-C₇ cycloalkyl, or a pharmaceutically acceptable salt thereof.

In some embodiments, wherein R¹ is methyl. In some embodiments, wherein R² is —CN. In some embodiments, R³ is fluoro. In some embodiments, R⁴ is C₁-C₆ alkyl. In some embodiments, R⁴ is ethyl. In some embodiments, the compound is of the formula

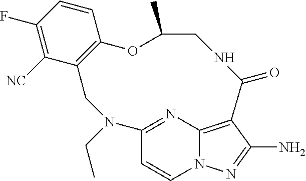 [SMILES: CCN1Cc2c(ccc(F)c2C#N)O[C@@H](C)CNC(=O)c2c(N)nn3ccc1nc23]

or a pharmaceutically acceptable salt thereof.

It will be appreciated that the disease can be any of a number of diseases associated with the tyrosine kinases described herein against which the compounds of the formula I have activity. For example, the methods described herein can be used for the treatment of diseases such as cancer, pain, psoriasis, rheumatoid arthritis, polycythemia vera, essential thrombocythemia, ulcerative colitis, myeloid metaplasia with myelofibrosis, and the like. It will be appreciated that the disease can be any disease associated with the activity of a genetically altered MET, a SRC, or CSF1R. In some embodiments, the disease is a cancer mediated by or associated with a genetically altered MET. In some embodiments, the disease is a cancer mediated by or associated with a genetically altered MET encoding a point mutation that is expressed in the c-Met protein. In some embodiments, the disease is a cancer mediated by or associated with a genetically altered MET encoding a point mutation expressed in the c-Met protein at one or more of positions P991, T992, V1092, H1094, G1163, T1173, L1195, F1200, D1228, Y1230, Y1235, D1246, Y1248, M1250, and M1268. In some embodiments, the disease is a cancer mediated by or associated with a genetically altered MET encoding a point mutation expressed in the c-Met protein that is selected from the group consisting of T1173I, P991S, M1250T, T992I, V1092I, F1200I, Y1235D, Y1230H, D1246N, D1246H, Y1248D, Y1248H, Y1248C, and M1268T.

It will be appreciated that the cancer can be any cancer mediated by or associated with a genetically altered MET, a SRC, or SCF1R including, but not limited to, a carcinoma, a sarcoma, a lymphoma, Hodgekin's disease, a melanoma, a mesothelioma, Burkitt's lymphoma, a nasopharyngeal carcinoma, a leukemia, a lung cancer, a breast cancer, a hereditary human papillary renal carcinoma, a sporadic human papillary renal carcinoma, a childhood hepatocellular carcinoma, or a myeloma. In some embodiments, the cancer includes, but is not limited to, ALCL, NSCLC, neuroblastoma, inflammatory myofibroblastic tumor, renal cancer, adult renal cell carcinoma, pediatric renal cell carcinoma, breast cancer, triple negative breast cancer, triple positive breast cancer, HER+breast cancer, mouth cancer, esophageal cancer, laryngeal cancer, pancreatic cancer, bladder cancer, colon cancer, colonic adenocarcinoma, glioblastoma, glioblastoma multiforme, thyroid cancer, anaplastic thyroid cancer, endocrine cancer, bone cancer, cholangiocarcinoma, ovarian cancer, cervical cancer, uterine cancer, testicular cancer, gastric cancer, gastric adenocarcinoma, colorectal cancer, rectal cancer, liver cancer, kidney cancer, angiosarcoma, epithelioid hemangioendothelioma, intrahepatic cholangiocarcinoma, thyroid papillary cancer, spitzoid neoplasms, sarcoma, astrocytoma, brain lower grade glioma, secretory breast carcinoma, mammary analogue carcinoma, acute myeloid leukemia, congenital mesoblastic nephroma, congenital fibrosarcomas, Ph-like acute lymphoblastic leukemia, thyroid carcinoma, skin cancer, head and neck squamous cell carcinoma, pediatric glioma CML, prostate cancer, lung squamous carcinoma, ovarian serous cystadenocarcinoma, skin cutaneous melanoma, metastatic castration-resistant prostate cancer, Hodgkin lymphoma, neuroendocrine tumors, serous and clear cell endometrial cancer

In some embodiments, the present disclosure provides methods of treating disease in a patient that has received a prior treatment with one or more therapeutic agents. In some embodiments, the patient has been previously treated with one or more therapeutic agents. In some embodiments, the patent has been previously treated with one or more therapeutic agents and developed an acquired resistance to the treatment. In some embodiments, the patent has been previously treated with one or more therapeutic agents and developed an acquired resistance to the treatment in the form of a genetically altered MET. In some embodiments, the patent has been previously treated with one or more therapeutic agents and developed an acquired resistance to the treatment expressed as a mutation of the c-Met protein encoded by a genetically altered MET. In some embodiments, the patent has been previously treated with one or more therapeutic agents and developed an acquired resistance to the treatment expressed as a point mutation in the c-Met protein at one or more of positions P991, T992, V1092, H1094, G1163, T1173, L1195, F1200, D1228, Y1230, Y1235, D1246, Y1248, M1250, and M1268. In some embodiments, the patent has been previously treated with one or more therapeutic agents and developed an acquired resistance to the treatment expressed as a point mutation selected from the group consisting of T1173I, P991S, M1250T, T992I, V1092I, F1200I, Y1235D, Y1230H, D1246N, D1246H, Y1248D, Y1248H, Y1248C, and M1268T in the c-Met protein that is encoded by a genetically altered MET. In some embodiments, the patent has been previously treated with one or more chemotherapeutic agents and developed bypass resistance to the treatment. In still other embodiments, the patent has been previously treated with one or more therapeutic agents and developed bypass resistance to the treatment regulated by SRC or SCF1R.

Other chemotherapeutic agents which the patient may be been treated with prior to treatment with one or more of the compounds described herein include but are not limited to kinase inhibitors, adrenocorticoids and corticosteroids, alkylating agents, peptide and peptidomimetic signal transduction inhibitors, antiandrogens, antiestrogens, androgens, aclamycin and aclamycin derivatives, estrogens, antimetabolites, platinum compounds, amanitins, plant alkaloids, mitomycins, discodermolides, microtubule inhibitors, epothilones, inflammatory and proinflammatory agents, purine analogs, pyrimidine analogs, camptothecins and dolastatins. In some embodiments, the chemotherapeutic agent the patient received previous to treatment with one or more compounds described herein can be one or more of afatinib, axitinib, alectinib, bosutinib, brigatini, cabozantinib, ceritinib, crizotinib, dabrafenib, dasatinib, erlotinib, everolimus, gefitinib, ibrutinib, imatinib, lapatinib, lenvatinib, nilotinib, nintedanib, palbociclib, pazopanib, ponatinib, regorafenib, ruxolitinib, sirolimus, sorafenib, sunitinib, tofacitinib, temsirolimus, trametinib, vandetanib, vemurafenib, methotrexate, busulfan, carboplatin, chlorambucil, cisplatin, tamoxiphen, taxol, paclitaxel, docetaxel, cytosine arabinoside, cyclophosphamide, daunomycin, rhizoxin, prednisone, hydroxyurea, teniposide, vincristine, vinblastine, eribulin, camptothecin, irinotecan, geldanamycin, estramustine and nocodazole. In some embodiments, the methods described herein provide treatment of a patient previously treated with a kinase inhibitor selected from the group consisting of afatinib, alectinib, axitinib, bosutinib, brigatini, cabozantinib, ceritinib, crizotinib, dabrefenib, dasatinib, erlotinib, everolimus, gefitinib, ibrutinib, imatinib, lapatinib, lenvatinib, nilotinib, nintedanib, palbociclib, pazopanib, ponatinib, regorafenib, ruxolitinib, sirolimus, sorafenib, sunitinib, tofacitinib, temsirolimus, trametinib, vandetanib and vemurafenib. In some embodiments, the patient was previously treated with crizotinib.

### Pharmaceutical Compositions

For treatment purposes, pharmaceutical compositions comprising the compounds described herein may further comprise one or more pharmaceutically-acceptable excipients. A pharmaceutically-acceptable excipient is a substance that is non-toxic and otherwise biologically suitable for administration to a subject. Such excipients facilitate administration of the compounds described herein and are compatible with the active ingredient. Examples of pharmaceutically-acceptable excipients include stabilizers, lubricants, surfactants, diluents, anti-oxidants, binders, coloring agents, bulking agents, emulsifiers, or taste-modifying agents. In preferred embodiments, pharmaceutical compositions according to the invention are sterile compositions. Pharmaceutical compositions may be prepared using compounding techniques known or that become available to those skilled in the art.

Sterile compositions are also contemplated by the invention, including compositions that are in accord with national and local regulations governing such compositions.

The pharmaceutical compositions and compounds described herein may be formulated as solutions, emulsions, suspensions, or dispersions in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. Pharmaceutical compositions of the invention may be administered by a suitable route of delivery, such as oral, parenteral, rectal, nasal, topical, or ocular routes, or by inhalation. Preferably, the compositions are formulated for intravenous or oral administration.

For oral administration, the compounds the invention may be provided in a solid form, such as a tablet or capsule, or as a solution, emulsion, or suspension. To prepare the oral compositions, the compounds of the invention may be formulated to yield a dosage of, e.g., from about 0.1 mg to 1 g daily, or about 1 mg to 50 mg daily, or about 50 to 250 mg daily, or about 250 mg to 1 g daily. Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid, or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil, such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions, or syrups, or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

For parenteral use, including intravenous, intramuscular, intraperitoneal, intranasal, or subcutaneous routes, the agents of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampoules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 g/kg/minute of agent admixed with a pharmaceutical carrier over a period ranging from several minutes to several days.

For nasal, inhaled, or oral administration, the inventive pharmaceutical compositions may be administered using, for example, a spray formulation also containing a suitable carrier. The inventive compositions may be formulated for rectal administration as a suppository.

For topical applications, the compounds of the present invention are preferably formulated as creams or ointments or a similar vehicle suitable for topical administration. For topical administration, the inventive compounds may be mixed with a pharmaceutical carrier at a concentration of about 0.10% to about 10% of drug to vehicle. Another mode of administering the agents of the invention may utilize a patch formulation to effect transdermal delivery.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³¹P, ³²P ³⁵S¹⁸F, ³⁶Cl, and ¹²⁵I, respectively. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

### Drug Combinations

The compounds described herein may be used in pharmaceutical compositions or methods in combination with one or more additional active ingredients in the treatment of the diseases and disorders described herein. Further additional active ingredients include other therapeutics or agents that mitigate adverse effects of therapies for the intended disease targets. Such combinations may serve to increase efficacy, ameliorate other disease symptoms, decrease one or more side effects, or decrease the required dose of an inventive compound. The additional active ingredients may be administered in a separate pharmaceutical composition from a compound of the present invention or may be included with a compound of the present invention in a single pharmaceutical composition. The additional active ingredients may be administered simultaneously with, prior to, or after administration of a compound of the present invention.

Combination agents include additional active ingredients are those that are known or discovered to be effective in treating the diseases and disorders described herein, including those active against another target associated with the disease. For example, compositions and formulations of the invention, as well as methods of treatment, can further comprise other drugs or pharmaceuticals, e.g., other active agents useful for treating or palliative for the target diseases or related symptoms or conditions.

Other chemotherapeutic agents suitable for use in combination in the methods described herein include but are not limited to kinase inhibitors, adrenocorticoids and corticosteroids, alkylating agents, peptide and peptidomimetic signal transduction inhibitors, antiandrogens, antiestrogens, androgens, aclamycin and aclamycin derivatives, estrogens, antimetabolites, platinum compounds, amanitins, plant alkaloids, mitomycins, discodermolides, microtubule inhibitors, epothilones, inflammatory and proinflammatory agents, purine analogs, pyrimidine analogs, camptothecins and dolastatins. In some embodiments, chemotherapeutic agents suitable for combination treatments in the methods described herein include but are not limited to one or more of afatinib, alectinib, axitinib, bosutinib, brigatini, cabozantinib, ceritinib, crizotinib, dabrefenib, dasatinib, erlotinib, everolimus, gefitinib, ibrutinib, imatinib, lapatinib, lenvatinib, nilotinib, nintedanib, palbociclib, pazopanib, ponatinib, regorafenib, ruxolitinib, sirolimus, sorafenib, sunitinib, tofacitinib, temsirolimus, trametinib, vandetanib, vemurafenib, methotrexate, busulfan, carboplatin, chlorambucil, cisplatin, tamoxiphen, taxol, paclitaxel, docetaxel, cytosine arabinoside, cyclophosphamide, daunomycin, rhizoxin, prednisone, hydroxyurea, teniposide, vincristine, vinblastine, eribulin, camptothecin, irinotecan, geldanamycin, estramustine and nocodazole. chemotherapeutic agents suitable for combination treatments in the methods described herein include but are not limited to one or more kinase inhibitor selected from the group consisting of afatinib, alectinib, axitinib, bosutinib, brigatini, cabozantinib, ceritinib, crizotinib, dabrefenib, dasatinib, erlotinib, everolimus, gefitinib, ibrutinib, imatinib, lapatinib, lenvatinib, nilotinib, nintedanib, palbociclib, pazopanib, ponatinib, regorafenib, ruxolitinib, sirolimus, sorafenib, sunitinib, tofacitinib, temsirolimus, trametinib, vandetanib and vemurafenib. In some embodiments, the patient was previously treated with crizotinib. For pain indications, suitable combination agents include anti-inflammatories such as NSAIDs. The pharmaceutical compositions of the invention may additional comprise one or more of such active agents, and methods of treatment may additionally comprise administering an effective amount of one or more of such active agents.

### Dosing and Administration

In some embodiments of the methods and compositions described herein, a therapeutically effective amount of one or more compounds that inhibits a genetically altered MET, SRC, and SCF1R, in particular a compound of the formula I, is administered to a host animal, such as a human patient, in need of treatment for cancer. In some embodiments of the methods and compositions described herein, a therapeutically effective amount of a compound that inhibits a genetically altered MET, SRC, and SCF1R, in particular Compound 1, is administered to a host animal, such as a human patient, in need of treatment for cancer.

As used herein, the term “therapeutically effective amount” refers to that amount of active compound or pharmaceutical agent that elicits the biological or medicinal response in a patient, which includes alleviation of the symptoms of the disease or disorder being treated. In one aspect, the therapeutically effective amount is that which may treat or alleviate the disease or symptoms. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors.

In some embodiments, an exemplary dose for a compound that inhibits a genetically altered MET, SRC, and SCF1R, in particular a compound of the formula I, more particularly Compound 1, in the various methods and compositions described herein is in the range of about from about 1 mg to about 3 g, or about 1 mg to about 500 mg, or about 50 to about 250 mg, or about 150 to about 500 mg, or about 150 to about 250 mg, or about 250 mg to about 1 g, or about 100 mg to about 2 g, or about 500 mg to about 2 g, or about 500 mg to about 1 g. It will be appreciated that all possible subranges within the dose ranges described above are contemplated and described herein. For example, a dose range of about 40 to about 500 mg for a compound that inhibits a genetically altered MET, SRC, and SCF1R, in particular a compound of the formula I, more particularly Compound 1, provided in the methods and compositions described herein includes doses of about 40 mg, about 50 mg, about 60, mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, including all possible doses and ranges as may be required based on such factors for determining a therapeutically effective amount as described herein. In some embodiments, the compound that inhibits a genetically altered MET, SRC, and SCF1R, in particular a compound of the formula I, more particularly Compound 1, provided in the methods and compositions described herein can be dosed at about 40 mg, about 80 mg, about 120 mg, about 160 mg, about 200 mg, about 240 mg, or about 280 mg.

In some embodiments, an exemplary dose for a compound that inhibits a genetically altered MET, SRC, and SCF1R, in particular a compound of the formula I, more particularly Compound 1, in the various methods and compositions described herein is in the range of about from about 1 mg to about 3 g daily, or about 1 mg to about 500 mg daily, or about 50 to about 250 mg daily, or about 150 to about 500 mg daily, or about 150 to about 250 mg daily, or about 250 mg to about 1 g daily, or about 100 mg to about 2 g daily, or about 500 mg to about 2 g daily, or about 500 mg to about 1 g daily. It will be appreciated that all possible subranges within the daily dose ranges described above are contemplated and described herein. For example, a dose range of about 40 to about 500 mg daily for a compound that inhibits a genetically altered MET, SRC, and SCF1R, in particular a compound of the formula I, more particularly Compound 1, provided in the methods and compositions described herein includes doses of about 40 mg daily, about 50 mg daily, about 60, mg daily, about 70 mg daily, about 80 mg daily, about 90 mg daily, about 100 mg daily, about 110 mg daily, about 120 mg daily, about 130 mg daily, about 140 mg daily, about 150 mg daily, about 160 mg daily, about 170 mg daily, about 180 mg daily, about 190 mg daily, about 200 mg daily, about 210 mg daily, about 220 mg daily, about 230 mg daily, about 240 mg daily, and about 250 mg daily, about 260 mg daily, about 270 mg daily, about 280 mg daily, about 290 mg daily, about 300 mg daily, about 310 mg daily, about 320 mg daily, about 330 mg daily, about 340 mg daily, about 350 mg daily, about 360 mg daily, about 370 mg daily, about 380 mg daily, about 390 mg daily, about 400 mg daily, about 410 mg daily, about 420 mg daily, about 430 mg daily, about 440 mg daily, about 450 mg daily, about 460 mg daily, about 470 mg daily, about 480 mg daily, about 490 mg daily, about 500 mg daily, including all possible doses and ranges as may be required based on such factors for determining a therapeutically effective amount as described herein. In some embodiments, the compound that inhibits a genetically altered MET, SRC, and SCF1R, in particular a compound of the formula I, more particularly Compound 1, provided in the methods and compositions described herein can be dosed at about 40 mg daily, about 80 mg daily, about 120 mg daily, about 160 mg daily, about 200 mg, about 240 mg, or about 280 mg.

In some embodiments, an alternative exemplary dose for a compound that inhibits a genetically altered MET, SRC, and SCF1R, in particular a compound of the formula I, more particularly Compound 1, provided in the various methods and compositions described herein is in the range of about from about 0.1 mg/kg to about 1 g/kg, or about 0.5 mg/kg to about 50 mg/kg, or about 0.5 mg/kg to about 25 mg/kg, or about 1.0 mg/kg to about 10 mg/kg, or about 1.0 mg/kg to about 5 mg/kg, or about 0.1 mg/kg to about 5 mg/kg, or about 0.1 mg/kg to about 1 mg/kg, or about 0.1 mg/kg to about 0.6 mg/kg. It will be appreciated that all possible subranges within the dose ranges described above are contemplated and described herein. For example, a dose range of about 1.0 mg/kg to about 10 mg/kg for a compound that inhibits a genetically altered MET, SRC, and SCF1R, in particular a compound of the formula I, more particularly Compound 1, provided in the methods and compositions described herein includes doses of about 1.0 mg/kg, about 2.0 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg, and about 10.0 mg/kg, including all possible doses and ranges as may be required based on such factors for determining a therapeutically effective amount as described herein.

In some embodiments, an alternative exemplary dose for a compound that inhibits a genetically altered MET, SRC, and SCF1R, in particular a compound of the formula I, more particularly Compound 1, provided in the various methods and compositions described herein is in the range of about from about 0.1 mg/kg to about 1 g/kg daily, or about 0.5 mg/kg to about 50 mg/kg daily, or about 0.5 mg/kg to about 25 mg/kg daily, or about 1.0 mg/kg to about 10 mg/kg daily, or about 1.0 mg/kg to about 5 mg/kg daily, or about 0.1 mg/kg to about 5 mg/kg daily, or about 0.1 mg/kg to about 1 mg/kg daily, or about 0.1 mg/kg to about 0.6 mg/kg daily. It will be appreciated that all possible subranges within the dose ranges described above are contemplated and described herein. For example, a dose range of about 1.0 mg/kg to about 10 mg/kg daily for a compound that inhibits a genetically altered MET, SRC, and SCF1R, in particular a compound of the formula I, more particularly Compound 1, provided in the methods and compositions described herein includes doses of about 1.0 mg/kg daily, about 2.0 mg/kg daily, about 3.0 mg/kg daily, about 4.0 mg/kg daily, about 5.0 mg/kg daily, about 6.0 mg/kg daily, about 7.0 mg/kg daily, about 8.0 mg/kg daily, about 9.0 mg/kg daily, and about 10.0 mg/kg daily, including all possible doses and ranges as may be required based on such factors for determining a therapeutically effective amount as described herein.

It will be appreciated that various dosing schedules for administration of a compound that inhibits a genetically altered MET, SRC, and SCF1R, in particular a compound of the formula I, more particularly Compound 1, can be applied to the methods and compositions described herein. It will be further appreciated that a dosing schedule for a compound administered in the various methods and compositions described herein can be defined by cycles of the dosing schedule, where such cycles are defined by the number of days of treatment, number of doses of the compound, the total dose of the compound, and the like. In some embodiments, a host animal, such as a human patient in need of treatment, can be administered a compound that inhibits a genetically altered MET, SRC, and SCF1R, in particular a compound of the formula I, more particularly Compound 1, for at least one cycle, for at least two cycles, for at least three cycles, for at least four cycles, and the like. Alternatively, in some embodiments, a host animal, such as a human patient in need of treatment, can be administered a compound that inhibits a genetically altered MET, SRC, and SCF1R, in particular a compound of the formula I, more particularly Compound 1, for from 1 to about 50 cycles, from 1 to about 25 cycles, from 1 to about 20 cycles, from 1 to about 10 cycles, and the like. It will be appreciate that, in some embodiments, a dosing schedule for a compound administered in the various methods and compositions described herein can include a holiday period during which no compound is administered, and such holiday period can be measured in days. In some embodiments, a dosing schedule for a compound administered in the various methods and compositions described herein can be defined by a number of cycles as described herein, followed by a holiday period, followed by another number of cycles as described herein.

In some embodiments, an exemplary dosing schedule for a compound that inhibits a genetically altered MET, SRC, and SCF1R, in particular a compound of the formula I, more particularly Compound 1, provided in the various methods and compositions described herein can include administration of a single daily dose (QD) or divided dosage units (e.g., BID (twice daily), TID (three times daily), QID (four times daily)). In some embodiments, a dosing schedule for a compound administered in the various methods and compositions described herein can vary within a cycle, such as a compound administered in the various methods and compositions described herein administered QD for a set number of days (e.g. QD for 1 day, 2 days, 3 days, 4 days, etc) followed by BID for a set number of days (e.g. BID for 1 day, 2 days, 3 days, 4 days, etc).

### Diagnostic Tests

In some embodiments, the present disclosure provides methods for treating disease in a patient previously identified as having a genetically altered MET. In some embodiments, the present disclosure provides methods for treating cancer in a patient previously identified as having a genetically altered MET. In some embodiments, the present disclosure provides methods for treating disease in a patient comprising (i) identifying a genetically altered MET in the patient, and (ii) administering to the patient a therapeutically effective amount of a compound useful in the treatment of such disease.

It will be appreciated that the diagnosing or identifying a patient as having a genetically altered MET can be accomplished by any number of diagnostic tests known to one of skill in the art. For example, such diagnostic tests include, but are not limited to, fluorescence in situ hybridization (FISH), polymerase chain reaction (PCR), immunohistochemistry (IHC), whole genome sequencing, next generation sequencing, circulating tumor cell, and the like. It will also be appreciated that any of the methods known in the art and applicable to diagnosing a patient or identifying a patient in connection with the present disclosure involve the transformation of a biological sample from one state of matter to another by direct modification, chemical synthesis, by direct non-covalent connection, or other known means, to provide a modified sample that can be used to determine whether the subject has or does not have a genetically altered MET. In some embodiments, “diagnosing” or “identifying” with respect to the disease state of a patient means applying a diagnostic test, such as FISH, PCR, IHC, or sequencing, to a biological sample obtained from the patient.

It will be appreciated that FISH is a test that “maps” the genetic material in a person's cells. This test can be used to visualize specific genes or portions of genes. FISH is a cytogenetic technique that uses fluorescent probes that bind to only those parts of the chromosome with a high degree of sequence complementarity. Such FISH tests can be used to identify a patient with a genetically altered MET by any method known in the art, and such test can be used in combination with the methods described herein as either a means of prior identification of a patient for treatment, or the concomitant identification of a patient for treatment.

It will be appreciated that IHC refers to the process of detecting antigens (e.g., proteins) in cells of a tissue section by exploiting the principle of antibodies binding specifically to antigens in biological tissues. Immunohistochemical staining is widely used in the diagnosis of abnormal cells such as those found in cancerous tumors. Specific molecular markers are characteristic of particular cellular events such as proliferation or cell death (apoptosis). Visualising an antibody-antigen interaction can be accomplished in a number of ways. In the most common instance, an antibody is conjugated to an enzyme, such as peroxidase, that can catalyse a color-producing reaction. Alternatively, the antibody can also be tagged to a fluorophore, such as fluorescein or rhodamine. Such IHC tests can be used to identify a patient with a genetically altered MET by any method known in the art, and such test can be used in combination with the methods described herein as either a means of prior identification of a patient for treatment, or the concomitant identification of a patient for treatment.

It will be appreciated that PCR refers to a technology in molecular biology used to amplify a single copy or a few copies of a piece of DNA across several orders of magnitude, generating thousands to millions of copies of a particular DNA sequence. Such PCR tests can be used to identify a patient with a genetically altered MET by any method known in the art, and such test can be used in combination with the methods described herein as either a means of prior identification of a patient for treatment, or the concomitant identification of a patient for treatment.

It will be appreciated that whole genome sequencing or next-generation sequencing refers to a process that determines the complete DNA sequence of an organism's genome at a single time. This entails sequencing all of an organism's chromosomal DNA as well as DNA contained in the mitochondria. Such whole genome sequencing tests can be used to identify a patient with a genetically altered MET by any method known in the art, and such test can be used in combination with the methods described herein as either a means of prior identification of a patient for treatment, or the concomitant identification of a patient for treatment.

### Examples

The examples and preparations provided below further illustrate and exemplify particular aspects of embodiments of the disclosure. It is to be understood that the scope of the present disclosure is not limited in any way by the scope of the following examples. Compounds of the formula I, in particular Compound 1, are disclosed in International Patent Publication No. WO2019/023417, and were prepared according to the methods described therein, and which is incorporated herein by reference in its entirety, in particular with respect to the preparation of compounds of the formula I, and specifically the preparation of Compound 1.

### In-Vitro Assays

### Materials and Methods

Compound 1 was tested toward MET and mutated MET proteins in Reaction Biology Corporation's HotSpot kinase assays (data in Table 1). The individual substrate for each kinase was prepared in freshly made Reaction Buffer with the subsequent addition of required cofactors if needed, followed by addition of the individual kinase and gentle mixing. Compound 1 in DMSO was added into the kinase reaction mixture utilizing acoustic technology (Echo 550), and then γ-[³³P]-ATP (specific activity 0.01 μCi/4L final) was delivered into the reaction mixture to initiate the reaction. The kinase reaction was incubated for 120 minutes at room temperature. Reactions were then spotted onto P81 ion exchange paper (Whatman #3698-915), which was washed extensively in 0.75% Phosphoric acid. The radioactive phosphorylated substrate remaining on the filter paper was measured. Compound 1 was tested in a 10-dose IC₅₀ mode with 3-fold serial dilution starting at 1 μM, and the control compound, staurosporine, was tested in both a 10-dose IC₅₀ mode with 4-fold serial dilution starting at 20 M, and a 10-dose IC₅₀ mode with 3-fold serial dilution starting at 0.1 μM. All the reactions were carried out in the presence of 10 M ATP concentration. Kinase activity data were expressed as the percent remaining kinase activity in test samples compared to vehicle (dimethyl sulfoxide) reactions. IC₅₀ values and curve fits were obtained using Prism4 Software (GraphPad).

The enzymatic kinase inhibitory activities of Compound 1 were evaluated in the radiolabeled kinase KinaseProfiler assays performed by Eurofins Pharma Discovery Services (data in Table 2). Full details of the assay for each kinase are available on the Eurofins website, or in the accompanying protocol document. An example of the assay is for MET(M1268T): human MET (M1268T) protein is incubated with 8 mM MOPS pH 7.0, 0.2 mM EDTA, 1 mM Na3VO₄, 5 mM sodium β-glycerophosphate, 250 μM KKKGQEEEYVFIE (SEQ ID NO: 1), 10 mM MgAcetate and [γ-³³P]-ATP (specific activity and concentration as required). The reaction is initiated by the addition of the Mg/ATP mix. After incubation for 40 minutes at room temperature, the reaction is stopped by the addition of phosphoric acid to a concentration of 0.5%, 10 μL of the reaction is then spotted onto a P30 filtermat and washed four times for 4 minutes in 0.425% phosphoric acid and once in methanol prior to drying and scintillation counting.

The enzymatic inhibitory activities of Compound 1 against MET and 12 mutated MET proteins at Reaction Biology Corporation using the radiolabeled HotSpot kinase assay platform (Table 1). Compound 1 had potent inhibition to MET proteins with a range of different mutations (Table 1). A small subset of the mutations tested (i.e. positions D1228X and Y1230) displayed the least potency of those tested. In the Eurofins KinaseProfiler biochemical assays, Compound 1 had potent inhibitory activities toward a range of mutant forms of MET (Table 2). Taken together, Compound 1 has potent inhibitory activities toward a range of mutated MET proteins.

TABLE 1

| Kinase | IC₅₀ (nM) |
|---|---|
| MET | 0.1 |
| MET (T1173I) | 3.1 |
| MET (P991S) | 11 |
| MET (M1250T) | 15 |
| MET (T992I) | 21 |
| MET (V1092I) | 31 |
| MET (F1200I) | 31 |
| MET (Y1235D) | 44 |
| MET (Y1230H) | 450 |
| MET (Y1230C) | 1000 |
| MET (Y1230D) | 1000 |
| MET (D1228N) | 1000 |
| MET (D1228H) | 1000 |
| MET (Y1230A) | 1000 |

TABLE 2

| Kinase | IC₅₀ (nM) |
|---|---|
| MET | 1.6 |
| MET (M1268T) | 1.6 |
| MET (Y1248H) | 12 |
| MET (Y1248C) | 119 |
| MET (D1246N) | 149 |
| MET (D1246H) | 192 |
| MET (Y1248D) | 481 |

Compound 1 was evaluated in a panel of Ba/F3 engineered cell models of MET and MET harboring resistance mutations. For creation of engineered cell lines, the TPR-MET fusion gene and its mutations T1173I, M1250T, H1094Y, G1163R, L1195V, D1228N and Y1230C were synthesized at GenScript and cloned into pCDH-CMV-MCS-EF1-Puro plasmid (System Biosciences, Inc), respectively. Ba/F3 engineered cells were generated by infecting Ba/F3 cells with lentivirus containing the related wild type or mutant genes. Ba/F3 engineered cells was selected in RPMI-1640 supplemented with 10% fetal bovine serum, 100 U/mL of penicillin, and 1 μg/mL puromycin solution, 10 ng/IL-3 (Life Technologies), followed by a further selection in the same medium without IL-3.

The cell potency of Compound 1 was measured using cell proliferations assay for Ba/F3 cells harboring TRP-MET constructs (Table 3). For cell proliferation assays, stable Ba/F3 cells were seeded in 384 well white plate followed by the addition of the test compounds. After 72 hours of incubation, cell proliferation was measured using CellTiter-Glo 2.0 luciferase-based ATP detection assay (Promega) following the manufacture's protocol. IC₅₀s were determined using GraphPad Prism software (GraphPad, Inc., San Diego, CA).

TABLE 3

| TPR-MET | Compound 1 IC₅₀ (nM) |
|---|---|
| Wild Type | <0.2 |
| T1173I | <0.2 |
| M1250T | <0.2 |
| H1094Y | <0.2 |
| G1163R | 19.8 ± 11.0 |
| L1195V | 23.7 ± 10.0 |
| D1228N | 1810 ± 210 |
| Y1230C | 1880 ± 390 |

Based on the dose-response analysis, the following values were also calculated: MET IC₉₅=0.2 nM, G1163R IC₉₀=171 nM, and L1195V IC₉₀=175 nM.

1. A method of treating cancer in a patient in need thereof, comprising administering to the patient a therapeutically effective amount of a compound that inhibits MET, SRC and CSF1R, wherein the cancer is mediated by a genetically altered MET, wherein the compound is of formula 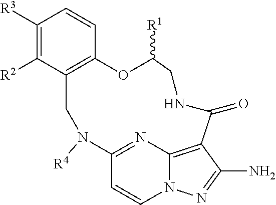
wherein
R¹ is H, deuterium, or C₁-C₆ alkyl;
R² is chloro or —CN;
R³ is H, deuterium, or fluoro;
R⁴ is H or C₁-C₆ alkyl, wherein each hydrogen atom in C₁-C₆ alkyl is independently optionally substituted by deuterium, fluoro, chloro, bromo, —OH, —CN, —OC₁-C₆ alkyl, —NH₂, —NH(C₁-C₆ alkyl), —N(C₁-C₆ alkyl)₂, or C₃-C₇ cycloalkyl, or a pharmaceutically acceptable salt thereof.

2. The method of claim 1, wherein the compound is of the formula 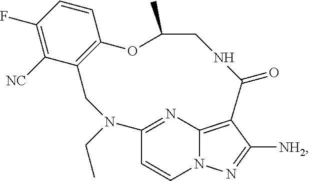 [SMILES: CCN1Cc2c(ccc(F)c2C#N)O[C@@H](C)CNC(=O)c2c(N)nn3ccc1nc23]
or a pharmaceutically acceptable salt thereof.

3. The method of claim 1, wherein the cancer is a carcinoma, a sarcoma, a lymphoma, Hodgekin's disease, a melanoma, a mesothelioma, Burkitt's lymphoma, a nasopharyngeal carcinoma, a leukemia, a lung cancer, a breast cancer, a hereditary human papillary renal carcinoma, a sporadic human papillary renal carcinoma, a childhood hepatocellular carcinoma, or a myeloma.

4. The method of claim 1, wherein the cancer is selected from the group consisting of ALCL, NSCLC, neuroblastoma, inflammatory myofibroblastic tumor, renal cancer, adult renal cell carcinoma, pediatric renal cell carcinoma, breast cancer, triple negative breast cancer, triple positive breast cancer, HER breast cancer, mouth cancer, esophageal cancer, laryngeal cancer, pancreatic cancer, bladder cancer, colon cancer, colonic adenocarcinoma, glioblastoma, glioblastoma multiforme, thyroid cancer, anaplastic thyroid cancer, endocrine cancer, bone cancer, cholangiocarcinoma, ovarian cancer, cervical cancer, uterine cancer, testicular cancer, gastric cancer, gastric adenocarcinoma, colorectal cancer, rectal cancer, liver cancer, kidney cancer, angiosarcoma, epithelioid hemangioendothelioma, intrahepatic cholangiocarcinoma, thyroid papillary cancer, spitzoid neoplasms, sarcoma, astrocytoma, brain lower grade glioma, secretory breast carcinoma, mammary analogue carcinoma, acute myeloid leukemia, congenital mesoblastic nephroma, congenital fibrosarcomas, Ph-like acute lymphoblastic leukemia, thyroid carcinoma, skin cancer, head and neck squamous cell carcinoma, pediatric glioma CML, prostate cancer, lung squamous carcinoma, ovarian serous cystadenocarcinoma, skin cutaneous melanoma, metastatic castration-resistant prostate cancer, Hodgkin lymphoma, neuroendocrine tumors, and serous and clear cell endometrial cancer.

5. The method of claim 1, wherein the patient has received prior treatment with one or more therapeutic agents.

6. The method of claim 1, wherein the cancer is mediated by a genetically altered MET.

7. The method of claim 6, wherein the genetically altered MET encodes a point mutation that is expressed in the c-Met protein.

8. The method of claim 7, wherein the genetically altered MET encodes a point mutation expressed in the c-Met protein at one or more of positions P991, T992, V1092, H1094, G1163, T1173, L1195, F1200, D1228, Y1230, Y1235, D1246, Y1248, M1250, and M1268.

9. The method of claim 8, wherein the genetically altered MET encodes a point mutation expressed in the c-Met protein that is selected from the group consisting of T1173I, P991S, M1250T, T992I, V1092I, F1200I, Y1235D, Y1230H, D1246N, D1246H, Y1248D, Y1248H, Y1248C, and M1268T.

10. The method of claim 9, wherein the cancer is exhibiting bypass resistance mediated by a SRC/CSF1R.

11.-20. (canceled)
